SEQUENCE LISTING (29 sequences)

SEQ ID NO: 1 - DNA, 1401 bp, Mus musculus
atggggaaacttcactcgaagccggccgccgtgtgcaagcgcagggagagcccggaaggtgacagctttgctgtaagcgctgcttgggcaaggaaaggcatcgaggagtggatcgggaggcagcgctgtccaggcagcgtctcaggaccccgtcagctgagattggcaggcactgttggtcgaggcactcgggaactcgtgggtgacacttctagagaggctctcggtgaggaggacgaggacgacttccccctagaagtggccctgccgcctgagaagatcgacagcctaggtagtggagatgagaagagaatggagagactgagcgaacctggccaggcctccaagaagcagctcaagtttgaagagctacagtgtgatgtctctgtggaggaggacagccggcaagagtggactttcactctatatgacttcgacaacaatggcaaagtgacccgtgaggacattaccagcttgctgcataccatctatgaagtggttgactcctctgtgaaccattcccccacatcaagcaagacactgcgggtgaagctcaccgtggctcctgacgggagccagagtaagaggagcgtccttttcaaccataccgatctgcagagcacaaggccccgagcagacaccaaacccgctgaggagctgcctggctgggagaagaagcagcgagccccactcaggttccagggtgacagccacctggagcagccagactgctaccaccattgcgtggatgagaacattgagaggagaaaccactacctagacctggcggggatagagaactacacgtctcagtttggaccgggatccccttcggtggcccagaagtcagagctgccccctcgaatctccaaccccactcgctctcgctcccacgagccagaagctgcccacatcccacaccggaggccccaaggtgtggacccaggctccttccacctccttgacaccccatttgccaaggcatcagagctccagcaacggctccggggcactcaggatgggagcaagcactttgtgaggtcccccaaggcccagggcaagaacatgggtatgggccacggggccagaggtgcaagaagcaagcctccactggtacccaccacccatactgtctccccctctgcccatctggccaccagcccagcccttctccccaccctggcacccctggggcacaagaaacacaagcatcgagccaaggagagccaggcgagctgccggggcctgcagggccccctggctgcaggaggctccaccgtcatggggcgggagcaggtgagggagctgcctgccgtggtggtgtacgagagccaggctgggcaggccgtccagagacacgaacaccatcaccaccacgaacatcaccaccattatcaccac

SEQ ID NO: 2 - protein, 472 aa, Mus musculus
MGKLHSKPAAVCKRRESPEGDSFAVSAAWARKGIEEWIGRQRCPGSVSGPRQLRLAGTVGRGTRELVGDTSREALGEEDEDDFPLEVALPPEKIDSLGSGDEKRMERLSEPGQASKKQLKFEELQCDVSVEEDSRQEWTFTLYDFDNNGKVTREDITSLLHTIYEVVDSSVNHSPTSSKTLRVKLTVAPDGSQSKRSVLFNHTDLQSTRPRADTKPAEELRGWEKKQRAPLRFQGDSHLEQPDCYHHCVDENIERRNHYLDLAGIENYTSQFGPGSPSVAQKSELPPRISNPTRSRSHEPEAAHIPHRRPQGVDPGSFHLLDTPFAKASELQQRLRGTQDGSKHFVRSPKAQGKNMGMGHGARGARSKPPLVPTTHTVSPSAHLATSPALLPTLAPLGHKKHKHRAKESQASCRGLQGPLAAGGSTVMGREQVRELPAVVVYESQAGQAVQRHEHHHHHHEHHHHYHHFYQP

SEQ ID NO: 3 - DNA, 2556 bp, Mus musculus
atgaaacccatgttgaaagacttttcaaatctcttgctggtggtgctctgtgactatgtcctcggagaagccgaatacctcctcctccaagagccagtccatgtggcactgagcgacagaacggtgtcagtgggtttccactacctcagtgacgtcaacgggacactgaggaatgtgtctgtcatgctgtgggaggccaacaccaatcggactcttaccactaagtacctcctgaccaaccaggcccaaggaacactccagtttgaatgtttctacttcaaagaggctggtgactactggtttgtaatgatcccggaagtgacagacaatggcacgcaagttccactctgggagaaaagtgcctttctgaaggtagaatggcctgtctttcacattgatttaaataggacagccaaggcagcagaaggcacctttcaagtgggtgtttttaccacccaaccgctctgcctgtttcccgtggacaagccagacatgctggtggatgttattttcactgaccgtcttccggaggcaagagcaagtttgggacagccgctggagatcagagccagcaaaaggacaaaactcactcaaggtcagtgggtcgagtttggctgtgcaccggtaggggtggaagcctacgttacagtcatgctgaggctgttgggtcaagactcagtcattgcttctacgggacctattgacctggctcaaaaattcggatacaaattgatgatggcaccggaagtcacgtgtgagtctgtgctggaggtgatggtactgccacctccttgtgtcttcgtccaaggagtcctggctgtttacaaagaagcccccaaacgcccggaggagaggactttccaggtggctgaaaacagactgcccctgggagagaggagaacggtgttcaactgcactttatttgatgtagggaagaacaaatactgttttaactttggaattgtgaagaaaggccatttttctgcaaaggaatgcatgctaattcagagaaatatagaaacttggggaccatggcagccgtggagcccgtgtagcaccacgtgcggggatgctgtccgagagcgtcgccgcctgtgtgtcacttctttcccctccagacccagctgctctggaatgtcctcagagacctctccatgctccctggaggagtgtgctgttttccggccaccaggcccatcgcctgtttcaccccaggaccctgtgaagtccaacaacgtggtgaccgtcacagggatctccctgtgcctgttcatcatctttgccacggtgctcatcactctctggaggaggtttggccgagcccccaaatgcagcacgcccgttcgccacaactccatccattcccctggcttccggaagaactccgatgaagagaacatctgcgagctgagtgagcctcgcggaagcttctcggatgccggtgacggacctaggggaagcccaggggacacgggcatcccattgacttacaggtgcagtgcatcagcgcctcctgaggatgaggcctcgggcagtgagagcttccagtccaacgctcagaagatcatcccgcccttgtttagctaccgccttgcccagcagcagctgaaggagatgaagaagaaagggctgaccgagaccaccaaagtgtaccacgtatctcaaagccccctgacagacactgtagtggatgccacggccagccctcccttagacctggaatgccccgaagaggctgcagcaagcaagttccgaatcaaatctccatttctggaccagcctggggcaggtaccggggaaaggcctccctccaggctggatggcatcgtgcctcctcctggctgtgcggtcagtcccagccagaccctgatccgaaagtcacagataaggtcgaccggtggcagagatggctcatcggagaggtgccactccagaagttccctcttcaggaggactgctagttttcatgaaaccaagcagtctcgccctttccgggagaggagtttgtcagccctgactccccgccaggtccccgcctacagttccaggatgcggacctgggaccagatggaggatagatgtcggcctcccagtcgaagtacccacctgcttccagagagaccagagcacttccaaggggcaggtcggaccagcagtcctttgggtccactctccaaatcctacactgtggggcatcccaggaggaaaccggacccaggggatcgtcaggccggattggtggcaggagctgagaaaatggagcctcaccgagctcacaggggaccgtcccccagtcacaggagtgcctcaaggaagcagtcttcccccattttcctcaaagatagctaccagaaagtcagtcagcttagcccttctcacttcagaaaagataaatgccagagcttccccatccaccccgagttcgccttctatgacaatacctctttccgcctcaccgaggctgagcagagaatgctggacctcccaggatacttcggctccaacgaagaggacgaaaccacaagtacactcagtgtggagaagttagtgatctag

SEQ ID NO: 4 - protein, 851 aa, Mus musculus
MKPMLKDFSNLLLVVLCDYVLGEAEYLLLQEPVHVALSDRTVSVGFHYLSDVNGTLRNVSVMLWEANTNRTLTTKYLLTNQAQGTLQFECFYFKEAGDYWFVMIPEVTDNGTQVPLWEKSAFLKVEWPVFHIDLNRTAKAAEGTFQVGVFTTQPLCLFPVDKPDMLVDVIFTDRLPEARASLGQPLEIRASKRTKLTQGQWVEFGCAPVGVEAYVTVMLRLLGQDSVIASTGPIDLAQKFGYKLMMAPEVTCESVLEVMVLPPPCVFVQGVLAVYKEAPKRPEERTFQVAENRLPLGERRTVFNCTLFDVGKNKYCFNFGIVKKGHFSAKECMLIQRNIETWGPWQPWSPCSTTCGDAVRERRRLCVTSFPSRPSCSGMSSETSPCSLEECAVFRPPGPSPVSPQDPVKSNNVVTVTGISLCLFIIFATVLITLWRRFGRAPKCSTPVRHNSIHSPGFRKNSDEENICELSEPRGSFSDAGDGPRGSPGDTGIPLTYRCSASAPPEDEASGSESFQSNAQKIIPPLFSYRLAQQQLKEMKKKGLTETTKVYHVSQSPLTDTVVDATASPPLDLECPEEAAASKFRIKSPFLDQPGAGTGERPPSRLDGIVPPPGCAVSPSQTLIRKSQIRSTGGRDGSSERCHSRSSLFRRTASFHETKQSRPFRERSLSALTPRQVPAYSSRMRTWDQMEDRCRPPSRSTHLLPERPEHFQGAGRTSSPLGPLSKSYTVGHPRRKPDPGDRQAGLVAGAEKMEPHRAHRGPSPSHRSASRKQSSPIFLKDSYQKVSQLSPSHFRKDKCQSFPIHPEFAFYDNTSFRLTEAEQRMLDLPGYFGSNEEDETTSTLSVEKLVI

SEQ ID NO: 5 - protein, 44 aa, Artificial Sequence
  Chemically synthesized oligopeptide.  Used to generate antibodies against DAP 1A
CETWGPWQPWSPCSTTCGDAVRERRRLCVTSFPSRPSCSGMSSE

SEQ ID NO: 6 - protein, 34 aa, Artificial Sequence
  Chemically synthesized oligopeptide.  Used to generate antibodies against DAP 1A
CRDGSSERCHSRSSLFRRTASFHETKQSRPFRER

SEQ ID NO: 7 - protein, 28 aa, Artificial Sequence
  Chemically synthesized oligopeptide.  Used to generate antibodies against DAP 1A
CRMRTWDQMEDRCRPPSRSTHLLPERPE

SEQ ID NO: 8 - protein, 26 aa, Artificial Sequence
  Chemically synthesized oligopeptide.  Used to generate antibodies against mNkd.
CRFQGDSHLEQPDCYHHCVDENIERR

SEQ ID NO: 9 - protein, 37 aa, Artificial Sequence
  Chemically synthesized oligopeptide.  Used to generate antibodies against mNkd.
CENYTSQFGPGSPSVAQKSELPPRISNPTRSRSHEPE

SEQ ID NO: 10 - protein, 22 aa, Artificial Sequence
  Chemically synthesized oligopeptide.  Used to generate antibodies against mNkd.
CRLRGTQDGSKHFVRSPKAQGK

SEQ ID NO: 11 - protein, 22 aa, Artificial Sequence
  Chemically synthesized oligopeptide.  Used to generate antibodies against mNkd.
CHKKHKHRAKESQASCRGLQGP

SEQ ID NO: 12 - DNA, 22 bp, Artificial Sequence
  Oligonucleotide. Used in PCR screen to amplify positive clones that contain mNkd sequence.
cctccaagaagcagctcaagtt

SEQ ID NO: 13 - DNA, 23 bp, Artificial Sequence
  Oligonucleotide. Used in PCR screen to amplify positive clones that contain mNkd sequence.
ttgtgctctgcagatcggtatgg

SEQ ID NO: 14 - DNA, 22 bp, Artificial Sequence
  Oligonucleotide. Used in PCR screen to amplify positive clones that contain DAP 1A sequence.
gaagaactccgatgaagagaac

SEQ ID NO: 15 - DNA, 22 bp, Artificial Sequence
  Oligonucleotide. Used in PCR screen to amplify positive clones that contain DAP 1A sequence.
gctttgagatacgtggtacact

SEQ ID NO: 16 - DNA, 27 bp, Artificial Sequence
  Oligonucleotide. Used in PCR to obtain 5′ end of DAP 1A.
cagcatgtctggcttgtccacgggaaa

SEQ ID NO: 17 - DNA, 27 bp, Artificial Sequence
  Oligonucleotide. Used in PCR to obtain 5′ end of mNkd.
cccgtcaggagccacggtgagcttcac

SEQ ID NO: 18 - DNA, 23 bp, Artificial Sequence
  Primer used in RT-PCR
tgtgaaccattcccccacatcaa

SEQ ID NO: 19 - DNA, 24 bp, Artificial Sequence
  Primer used in RT-PCR
aaatggggtgtcaaggaggtggaa

SEQ ID NO: 20 - protein, 668 aa, Drosophila melanogaster
MAGNIVKWWKHKILGGYKQFSVQECTTDSEELMYHQVRASSSCSAPPDLLLVSERDNNIQLRSPVVNIITTPPGNASGAGSKQQSHHQTNHHSSGRSHPGHTAHPQDVSSGGSHSKHLRISSTSNGKHGKYSNMQQQLPQDEDVVDAAATMQQQQHTGHAHSRHLHHHKEERIRLEEFTCDVSVEGGKSSQPLQFSFTFYDLDGHHGKITKDDIVGIVYTIYESIGKSVVVPHCGSKTINVRLTVSPEGKSKSQPVVPVPVAAGFSSSHASKLKKLPTGLAAMSKPLAGGGVGSGGASALTTSAGNRRQHRYRPRKLIKSDDEDDDSNSEKEKDAAHAPAADQPSGSGTKATGKSHHHQSQSARYHQKNNSRAEQCCTEQNTPDNGHNTYENMLNLKCCKPEVDQVDCPSHRQHHQSHPNHQMRQQDIYMKQATQRVKMLRRARKQKYQDHCLETRQRSLSVGNDSACPNRHLQLQQPPVGHPQPQSLNHKSASGSPPLGVGGGGDMMLDGVQLRQPRPHSLTPQQHQQQNQQQQQQQRKSAECWKSALNRNDLISIIRESMEKNRLCFQLNGKPQANVSPIRQPAAQQQPQQQQRQRCNTGSKIPTLITNHSPVAQQSPLSCSPPTAEPTTPSIPAAPPAIEVNGQQHHPTHPTHPSHHNHHEHPQP

SEQ ID NO: 21 - protein, 56 aa, Mus musculus
LKFEELQCDVSVEEDSRQEWTFTLYDFDNNGKVTREDITSLLHTIYEVVDSSVNHS

SEQ ID NO: 22 - protein, 60 aa, Drosophila melanogaster
IRLEEFTCDVSVEGGKSSQPLQFSFTFYDLDGHHGKITKDDIVGIVYTIYESIGKSVVVP

SEQ ID NO: 23 - protein, 60 aa, Homo sapiens
LDFKEYVIALHMTTAGKTNQKLEWAFSLYDVDGNGTISKNEVLEIVMAIFKMITPEDVKL

SEQ ID NO: 24 - protein, 60 aa, Drosophila melanogaster
IEFEEFIRALSVTSKGNLDEKLQWAFRLYDVDNDGYITREEMYNIVDAIYQMVGQQPQSE

SEQ ID NO: 25 - protein, 36 aa, Mus musculus
DSRQEWTFTLYDFDNNGKVTREDITSLLHTIYEVVD

SEQ ID NO: 26 - protein, 36 aa, Artificial Sequence
  mNkd sequence with EF-hand calcium bindind loop mutated.
DSRQEWTFTLYVFVNNGKVTREDITSLLHTIYEVVD

SEQ ID NO: 27 - protein, 36 aa, Artificial Sequence
  mNkd sequence with EF-hand calcium bindind loop mutated.
DSRQEWTFTLYDFDNNWKVTREDITSLLHTIYEVVD

SEQ ID NO: 28 - protein, 36 aa, Artificial Sequence
  mNkd sequence with EF-hand calcium bindind loop mutated.
DSRQEWTFTLYDFDNNGKKTREDITSLLHTIYEVVD

SEQ ID NO: 29 - protein, 10 aa, Artificial Sequence
  mNkd sequence with EF-hand calcium bindind loop deleted.
DSRQEYEVVD

(19) United States
(12) Patent Application Publication
(10) Pub. No.: US 2002/0061552 A1
(43) Pub. Date: May 23, 2002

(54) Mammalian dishevelled-associated proteins

(76) Inventors: Dong Yan, Emeryville, CA (US); Lewis T. Williams, Mill Valley, CA (US)

Correspondence Address: Chiron Corporation, Intellectual Property - R440, P.O. Box 8097, Emeryville, CA, 94662-8097, US

(21) Appl. No.: 09/730,989

(22) Filed: Dec. 5, 2000

Related U.S. Application Data
(60) Provisional application No. 60/172,434, filed on Dec. 17, 1999.

(51) Int. Cl. A61K 38/00; C07H 21/04; C12P 21/06; C12N 15/00; C12N 15/09; C12N 15/63; C12N 15/70; C12N 15/74; C12N 5/00; C12N 5/02; C07K 5/00; C07K 7/00; C07K 16/00; C07K 17/00; A61K 38/04; C07K 1/00; C07K 14/00; C12N 15/85; C12N 15/87

(52) U.S. Cl. 435/69.1; 435/325; 435/455; 435/320.1; 536/23.5; 530/387.1; 530/350; 530/326; 514/13

(57) ABSTRACT

Two novel proteins that interact with mammalian Dishevelled protein, and the corresponding polynucleotide sequences encoding the proteins, are disclosed. The proteins are referred to as mNkd and DAP 1A. mNkd is expressed at a higher level in mammalian lung tissues than in other mammalian tissues. mNkd inhibits Wnt signaling, and is an activator of the JNK pathway.

### CROSS-REFERENCE TO RELATED APPLICATIONS

This application claims priority from U.S. patent application No. 60/172,434 filed Dec. 17, 1999, which is incorporated by reference herein in its entirety.

### TECHNICAL FIELD

The invention relates to genes encoding proteins involved in the Wnt signaling pathway, to fragments of the proteins, and to methods of using the genes and gene products.

### BACKGROUND OF THE INVENTION

A Drosophila gene referred to as Dishevelled (Dsh) encodes a protein which is a component in a chain of proteins that carry the wingless signal from cell membrane to nucleus. Dsh is well conserved in relation to its vertebrate homologs. All Dsh studied to date have three highly conserved domains. The N-terminal DIX domain is also present in Axin, a negative regulator of wingless signaling. The internal PDZ domain has been shown to be a protein-protein interactive domain. The DEP domain has been implicated in G protein signaling. In addition to being instrumental to the wingless pathway, Dsh is also required in the planar polarity pathway in Drosophila, where it activates Jun Terminal Kinase (JNK). Several lines of evidence indicate that Dsh is differentially recruited into these two different pathways. The third known function of Dsh is that it interacts with Notch, and possibly blocks Notch signaling.

Wg/Wnt ligands and their receptors frizzled are involved in at least two pathways. One pathway is via the β-catenin route and determines cell growth, development and oncogenesis. The other goes through Rho and c-jun N-terminal kinase to establish planar polarity in epidermal structures. Dishevelled is a proximal downstream component required in both pathways. Extensive genetic and biochemical studies on the roles of Dishevelled in the two pathways have identified that the DIX and PDZ domains are necessary for Wnt/β-catenin signaling, while the DEP domain is required in determining planar polarity (Boutros and Mlodzik, Mech. Dev. 83:27, 1999).

Although the exact function of Dishevelled in higher organisms remains to be determined, a strain of mice with mouse Dishevelled 1 (mDv11) deficiency exhibits characteristics of some neurological disorders in humans (Cell 90:895-905, 1997). This strain provides a model for further studying the roles of the gene in mice. Further understanding of the functions of Dsh in the wingless, JNK, and notch pathways will be expedited by the discovery of proteins that are physically or functionally related to Dsh.

### SUMMARY OF THE INVENTION

The invention relates to a novel mammalian protein that associates with the dishevelled protein, and is named mNkd.

The invention relates to a second novel mammalian protein that associates with the dishevelled protein, and is named DAP (dishevelled-associated protein) 1A.

The invention further relates to polynucleotides encoding mNkd and DAP 1A.

The invention also relates to variants and homologs of the polynucleotides encoding mNkd and DAP 1A.

The invention still further relates to proteins sharing the biological function of mNkd or DAP 1A, but having at least one amino acid substitution, addition, or deletion relative to corresponding native mNkd or DAP 1A.

The invention also relates to fragments of mNkd and DAP 1A, wherein the fragments retain at least one biological activity of the native proteins.

The invention further relates to antibodies capable of specifically binding to at least one of the proteins mNkd and DAP 1A.

The invention still further relates to a complex comprising a dishevelled protein or a fragment thereof, and at least one of the proteins mNkd and DAP 1A, or a fragment thereof capable of binding to the dishevelled protein or fragment of the dishevelled protein.

The invention also relates to a method of activating the JNK pathway using mNkd.

The invention still further relates to a method of inhibiting Wnt signaling in a mammalian cell by overexpressing mNkd in the mammalian cell.

The invention also relates to agonists and antagonists of these two proteins, knock-outs of these two genes, gene therapy, antisense and ribozymes that target DAP 1A and mNkd mRNA, and antibodies.

The invention further relates to an isolated nucleic acid molecule comprising a polynucleotide selected from the group consisting of: (a) a polynucleotide encoding amino acids from about 1 to about 460 of SEQ ID NO:2; (b) a polynucleotide encoding amino acids from about 2 to about 460 of SEQ ID NO:2; (c) a polynucleotide encoding amino acids from about 1 to about 820 of SEQ ID NO:4; (d) a polynucleotide encoding amino acids from about 2 to about 820 of SEQ ID NO:4 ; (e) the polynucleotide complement of the polynucleotide of (a), (b), (c), or (d); and (f) a polynucleotide at least 90% identical to the polynucleotide of (a), (b), (c), (d), or (e).

The invention also relates to an isolated polypeptide comprising amino acids at least 95% identical to amino acids selected from the group consisting of: (a) amino acids from about 1 to about 460 of SEQ ID NO:2; (b) amino acids from about 2 to about 460 of SEQ ID NO:2; (c) amino acids from about 1 to about 820 of SEQ ID NO:4; and (d) amino acids from about 2 to about 820 of SEQ ID NO:4, and to antibodies capable of binding to these polypeptides.

### BRIEF DESCRIPTION OF THE FIGURES

FIG. 1 illustrates the full length sequence of mNkd polynucleotide (SEQ ID NO:1).

FIG. 2 illustrates the full length sequence of mNkd protein (SEQ ID NO:2).

FIG. 3 illustrates the full length sequence of DAP 1A polynucleotide (SEQ ID NO:3).

FIG. 4 illustrates the full length sequence of DAP 1A protein (SEQ ID NO:4).

FIG. 5 illustrates that mNkd inhibits Wnt-1 activated LEF-1 luciferase reporter. 293 cells were transfected with constructs expressing either Wnt-1 alone (W.1/GFP) or Wnt-1 together with mNkd (W.1/10C). Full length mNkd can inhibit Wnt-1 induced activation of the LEF-1 luciferase reporter. However, expression of only the Dvl binding domain of mNkd (W.1/10CBD) did not inhibit Wnt-l induced activity.

FIG. 6 illustrates that mutations in the EF hand region of mNkd affect its function. The constructs are as follows: Vector.3, Wnt.1/10V.2, Wnt.1/10.2, Wnt.1/m2.2, Wnt.1/m3.2, Wnt.1/10Cm4.2, and Wnt.1/10BD.2, all of which are in pcDNA3.1 HisC vectors.

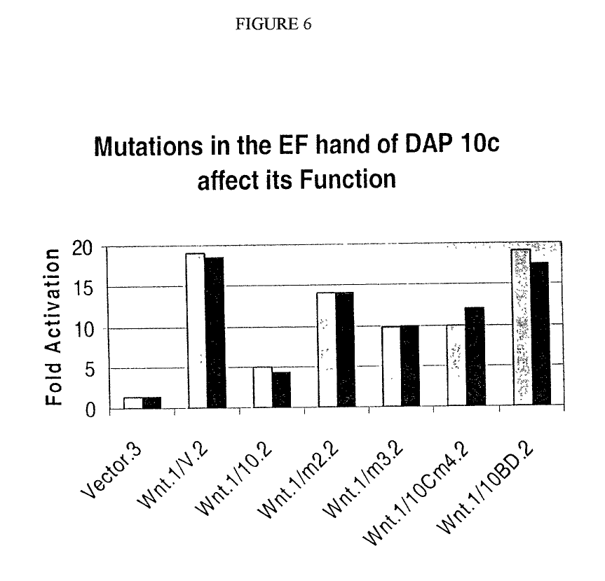

FIG. 7 illustrates that mNkd activates JNK in NIH 3T3 cells. NIH 3T3 cells were transfected with increasing amounts of mNkd. The membrane was blotted with anti-phospho c-Jun II antibody, which specifically recognizes the phosphorylated serine at position 63 in the N-terminus of c-Jun. The same membrane was then stripped and blotted with anti-x-press antibody, which recognizes the expressed mNkd and β-Gal (to normalize the amount of DNA transfected). The amount of protein in each sample was indicated by the signal of GAP on the same membrane.

FIG. 8(A) provides an amino acid sequence alignment of mNkd with Drosophila Nkd. Deduced amino acid sequences for mNkd and Nkd (Zeng et al., Nature 403:789, 2000) were compared using the Macvector ClusterW program. The EF-hand motif in each protein is underlined. Identical amino acids are highlighted in gray and the conserved changes are highlighted in light gray. (B) Alignments are provided of the EF-hand motifs from mNkd, Nkd, human Recoverin, and Drosophila Frequenin. Amino acids which are identical in more than two EF-hand motifs are highlighted in gray. The conserved changes are highlighted in light gray.

FIG. 9 shows a section of the mNdk protein having amino acid substitutions.

FIG. 10 illustrates the relative location of the DIX, PDZ and DEP regions of Dsh.

FIG. 11 is a bar graph illustrating the effects of mNkd and its EF-hand mutants on the Wnt responsive receptor activities.

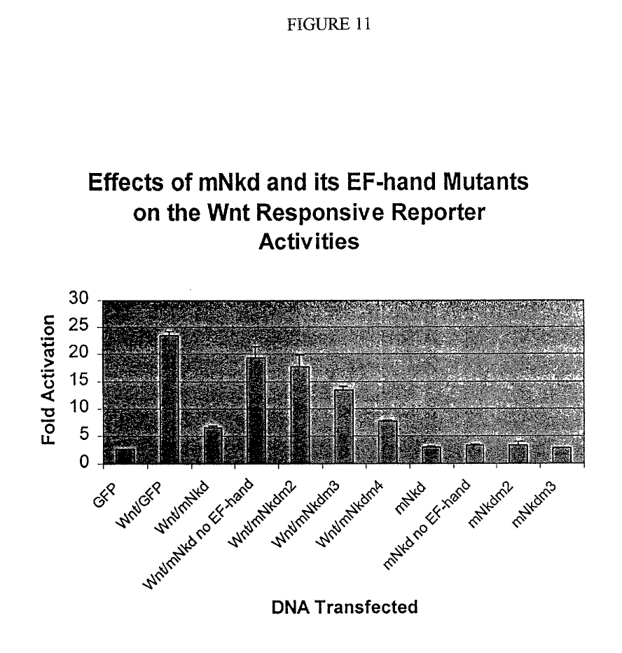

FIG. 12 is a bar graph illustrating the effects of mNkd on β-catenin activated reporter.

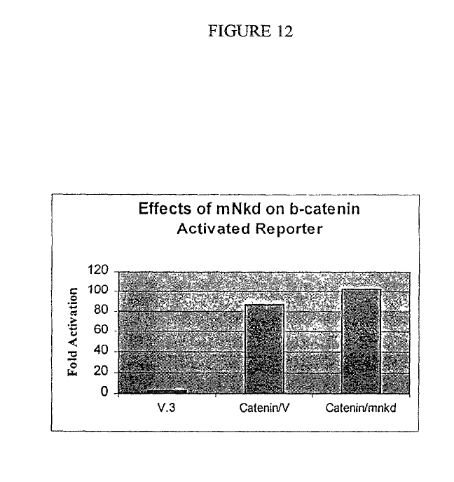

FIG. 13 illustrates secondary axis formation in Xenopus embryos. Ventral injection of 5-10 pg of XWnt-8 mRNA induced secondary axes in over 60% of the embryos, and injection of mNkd mRNA suppressed the activity of XWnt-8.

### DETAILED DESCRIPTION OF THE INVENTION

The Wg[Wnt signaling pathway is regulated by positive and negative effectors. Recently, a gene referred to as nkd was described in Drosophila, and the gene encodes a Wg-inducible inhibitor of Wg signaling (Zeng et al., Nature 403:789, 2000). The mechanism by which this inhibition occurs remains unknown. Drosphila nkd is a structural and functional homologue of mammalian Nkd, mNkd, whose mRNA levels increase in response to Wnt and which is the subject of the present invention. According to the invention, mNkd antagonizes the Wnt pathway by blocking the effects of Wnt on 13-catenin in both cell culture and vertebrate Xenopus laevis. Further, mNkd also affects JNK planar polarity pathway in these systems. These effects appear to be mediated by a direct interaction of mNkd with Dishevelled, a common component of both the Wnt and planar polarity pathways.

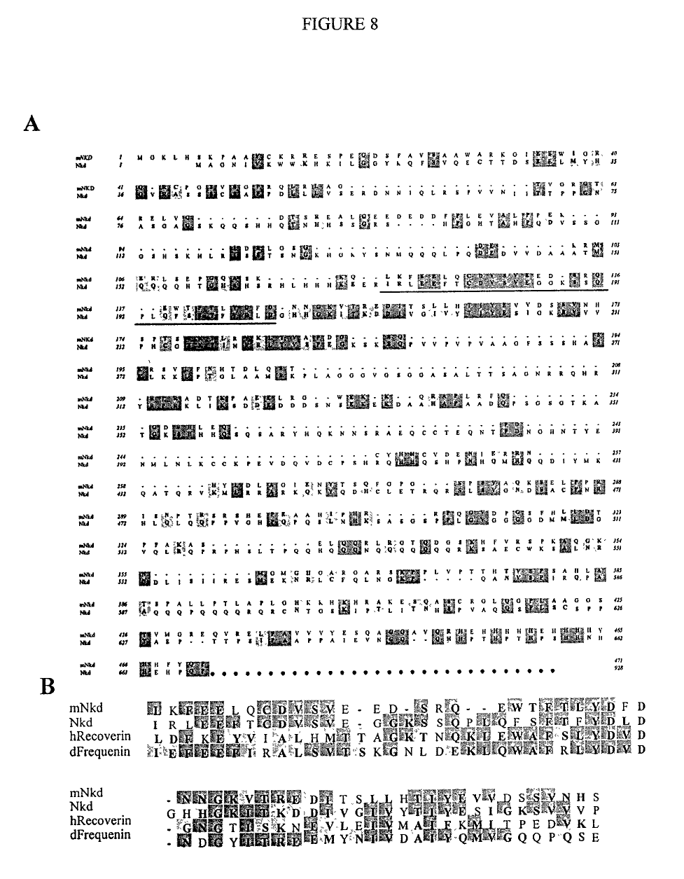

It was recently shown that Nkd antagonizes the Wg/Wnt pathway (Zeng et al., 2000). However, little is known about the mechanism for the effect. As disclosed herein, in order to identify additional Dvl associated proteins that may function in both pathways, a mouse embryonic 9.5 and 10.5 d.p.c. library was screened using a yeast two-hybrid approach. Several protein fragments were identified as being able to interact with the full-length mouse Dv12 and Dv13. One of the novel proteins contains a single EF-hand calcium-binding motif. This protein has now been shown to be 49% similar and 34% identical to the recently reported Drosophila Nkd (FIG. 8A) that also contains a very similar EF-hand (FIG. 8B). We have now named the protein mNkd (for mouse Nkd). The domain of mNkd that interacts with Dvl in the two-hybrid is located between amino acids 107 to 230, including the EF-hand motif. Using mNkd as a query to search the Genbank database, Nkd was found to be the most closely related protein (P=4E-12) in Drosophila. Conversely, using Nkd in a query to search a mouse EST database, a few partial mNkd fragments were identified to be most closely related to the Drosophila Nkd.

To determine the expression pattern of mNkd in mammalian tissues, Northern analyses were performed with RNA blots of mouse adult tissues and E7 to E17 embryos (Clontech) using mNkd as a probe. In adult mice, a transcript of 1.7-kb was detected at high level only in lung and liver. This 1.7-kb transcript matches with the size of the cloned mNkd cDNA. This transcript was detected at lower levels in heart, brain and testis. In addition, the probe detected two weak bands at 3.0-kb and 4.4-kb in adult tissues. In mouse embryos, a major 1.7-kb and two minor 3.0-kb and 4.4-kb transcripts were detected at all stages of development. It is likely that the 3.0-kb and 4.4-kb transcripts may represent other isoforms of the protein.

To confirm the interactions of mNkd and Dvl in mammalian cells, we examined if mNkd could interact with endogenous Dvl. Xpress tagged mNkd was transiently expressed in Cos7 cells and total cell lysate was immunoprecipitated with antibodies against Dvl 1, 2, and 3. Xpress-mNkd was detected in the Dvl immunocomplex. To study the requirement of the mNkd EF-hand for binding to Dvl in vivo, a large part of this domain was deleted (amino acids 138 to 163). The resulting construct (mNkd A EF) was expressed in Cos7 cells. Total cell lysate was immunoprecipitated with antibodies against endogenous Dvl. mNkd A EF was detected in the Dvl immuno-complexes by Western blotting. The same result was also obtained in HEK293 cells. Single or double mutations within the EF-hand also appeared to have no detectable effect on the binding of these mutants to Dvl. These results indicate that mNkd is associated with Dvl in cell lysate and the association does not require the EF-hand.

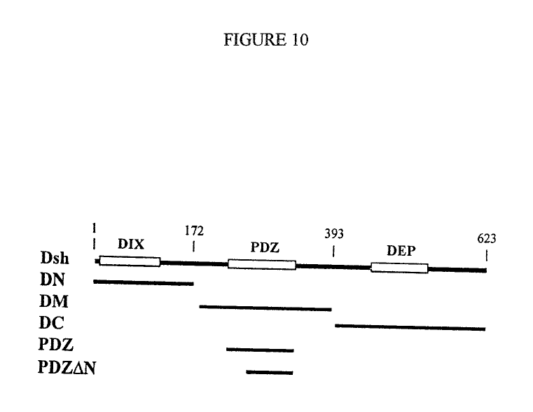

Some positive and negative regulators of the Wnt pathway, including FRATI, CKlε, and Axin, bind to Dvl at positions that can be shared or separated. This binding pattern could provide a possible mechanism for regulation of the Wnt pathway. Thus, it was of interest to determine which region of Dvl mNkd was bound to. Fragments corresponding to different regions of Drosophila Dvl (FIG. 10) were expressed in E. coli as GST fusion proteins. Equal amounts of each fragment were mixed with in vitro translated mNkd in binding buffer and separated on a Tris-Glycine gel. mNkd associated with only the DM fragment of Dvl which contains the PDZ domain and the basic amino acids stretch immediately before the PDZ domain. The PDZ domain alone was not sufficient for the binding to mNkd. Neither the N-terminal nor the C-terminal domain of Dvl can bind to mNkd. This results showed that mNkd is associated with a region on Dsh that is shared with FRATI (Yost et al., Cell 93:1031, 1998; Li et al., EMBO Jour. 18:4233, 1999) and CKlε (Peters et al., Nature 401:345, 1999; Sakanaka et al., Proc. Natl. Acad. Sci. 96:12548, 1999), both of which play a role in β-catenin stability.

Since Dvl mediates the Wnt signal, the role of mNkd was investigated in the Wnt pathway. It has been reported previously that Wnt-mediated activation of the pathway in mammalian cells can be measured using a LEF-1 readout system (Hsu et al., Mol. Cell. Biol. 18:4807, 1988). Expression of either mNkd alone or EF-hand deletion mutant mNkd ΔEF alone in HEK 293 cells had no effect on the reporter readout. However, when mNkd was co-expressed with Wnt, the well-documented activation of the reporter by Wnt was inhibited by about 75% in multiple experiments. These data suggest that mNkd negatively regulates the Wnt signaling, a result similar to the antagonistic effect of Drosophila Nkd on Wg. Interestingly, mNkdΔ EF only inhibited approximately 20% of the Wnt response. Furthermore, mNkd with point mutations within the EF-hand also failed to inhibit Wnt signal at a level as well as wild-type mNkd did. Taken together, these data imply that the EF-hand is essential for mNkd to inhibit Wnt signaling. The failure of these EF-hand mutations in inhibiting Wnt signaling is not due to their binding abilities to Dvl, since these mutants all appeared to bind to Dvl at no significant difference from the wild-type mNkd. Furthermore, these results also argue against the possibility that the inhibitory effect of mNkd to the Wnt signaling in the cell culture assay is due to sequestering of Dvl from the pathway.

Expression of β-catenin also activates the LEF-1 reporter. However, as shown by experiments described in the Examples, this activation could not be inhibited by co-expression of mNkd. These results indicate that mNkd inhibits Wnt response at a step upstream of β-catenin.

Since mNkd can inhibit Wnt signaling in cell culture, its function in vertebrate Xenopus laevis was examined. mNkd mRNA was injected into Xenopus embryos and was found to inhibit Wnt induced secondary axis formation.

mNkd expression was examined in cells treated with media containing Wnt ligand or no Wnt ligand. When cultured mammalian cells were treated with Wnt-3A conditioned medium or control medium for periods of 8 hrs., 19.5 hrs., or 27 hrs., mNkd transcripts were significantly increased after 19.5 hrs. or 27 hrs. of treatment as detected by RT-PCR. Control medium treatment for the same length of time had no effect on mNkd transcription. Wnt-3A conditioned medium treatment for 8 hrs. induced significantly less mNkd than the 19.5 hrs. or 27 hrs. treatment. The level of GAPDH transcripts in each sample was not altered by the treatment. Similar induction effect was also seen in L cells treated with Wnt, although the effect is less potent. Lithium chloride treatment of the same cells for 16 hrs. also strongly induced mNkd transcription. These data indicate that mNkd may be a direct target of Wnt signaling and may be involved in feedback inhibitory regulation of the pathway.

Since Dvl functions at a branchpoint that can lead to either the Wnt/β-catenin or the JNK planar polarity pathways, experiments were performed to determine whether mNkd had any effect on the JNK planar polarity pathway. mNkd expressed in NIH 3T3 cells activated JNK, as shown by increased signals when cell lysate was blotted with a phospho-c-Jun antibody (New England Biolab). These data reveal that while mNkd is involved in the Wnt pathway, it may also play a role in determining planar polarity.

Inhibition of Dvl function in Xenopus resulted in convergent extension defect during gastrulation. This convergent extension defect is a result of cell polarity abnormality caused by defects in Dvl function. Ectopic expression of mNkd in Xenopus inhibited convergent extension.

Loss of function of negative regulators of the Wnt pathway is one of the mechanisms that underlies Wnt pathway involvement in oncogenesis in mammals. The invention provides a mouse homologue of Drosophila Nkd that shares not only the sequence similarity, but also shares the functional similarity with Nkd. Since mNkd inhibited Wnt signaling in both cell culture and Xenopus assays, and the transcription of mNkd is Wnt inducible, mNkd is likely to be involved in negative feedback of the pathway. Therefore, loss of function of mNkd can lead to excessive activation of the Wnt pathway in mammals.

Since the present data indicate that mNkd binds to Dvl directly and inhibits Wnt signaling upstream of β-catenin, the mechanism by which mNkd inhibit Wnt signaling could be by interacting with downstream components of the Wnt pathway at a step around Dvl. Activation of the Wnt pathway may lead to the increased expression of mNkd which then displaces other positive effectors from Dvl and leads to the inhibition of the pathway. The roles of mNkd in determining planar polarity may also be a result of mNkd binding to Dishevelled. The data disclosed herein also indicated the importance of the EF-hand calcium binding domain in regulating Wnt signaling, which may have correlation with the calcium regulation by the Wnt pathway.

In summary, the biological properties of the proteins of the invention are consistent with a role in the Wnt and JNK pathways. Specifically, as described in detail in the Examples, over-expression of mNkd inhibited Wnt signaling in mammalian cells. In addition, expression of mNkd in mammalian cells activated JNK, a response also seen by expression of Dsh. This suggests that mNkd is also an activator of the JNK pathway.

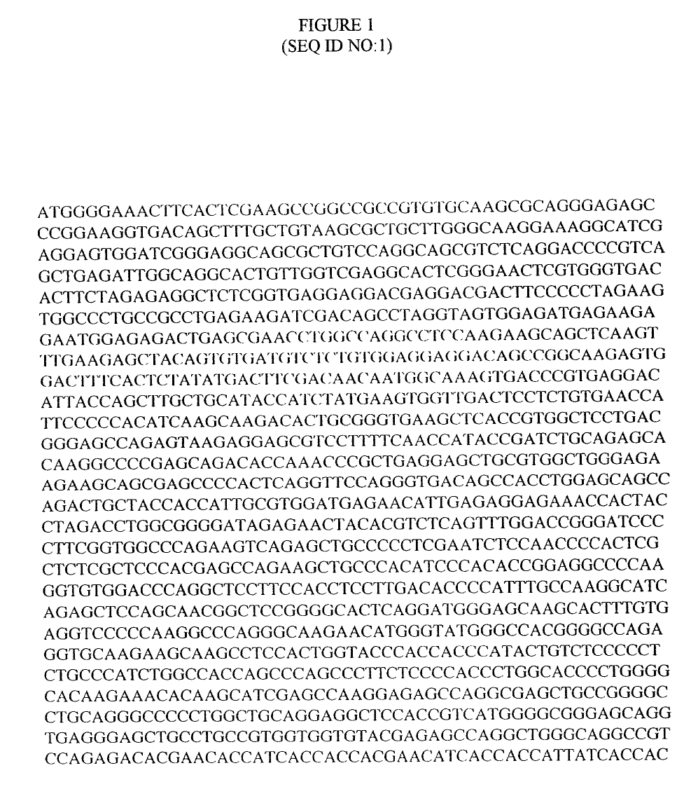

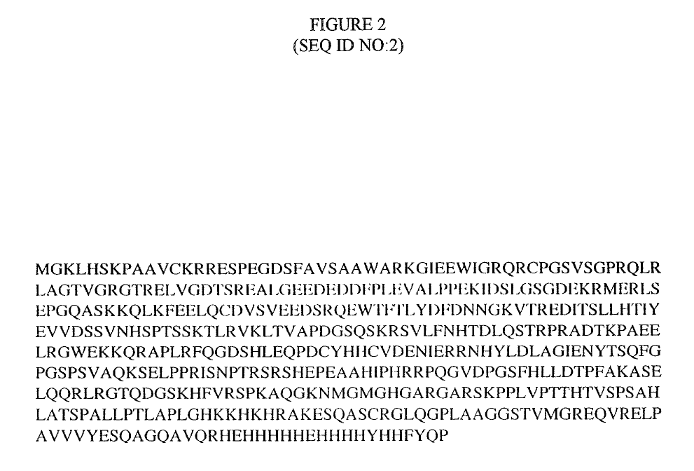

mNkd has a molecular weight of 52 kd and is encoded by a polynucleotide of 1416 basepairs. The polynucleotide and amino acid sequences are shown in FIGS. 1 and 2, as SEQ ID NO:1 and 2, respectively. mNkd contains a region of 29 amino acids encoded by nucleotides 406-489, which is highly homologous to the EF hand of calcium binding proteins. This region is within the part of the mNkd protein that interacts with Dv13.

The EF hand region of mNkd plays a role in the inhibitory effect of mNkd on Wnt signaling, as mutations in conserved amino acids within the EF hand region alleviate the inhibitory effect. One mNkd mutant (m2) was constructed by changing nucleotides A431 to T and A437 to T, resulting in changing amino acids D144 and D146 to V144 and V146. A second mNkd mutant (m3) was constructed by changing nucleotides G445 to T and G447 to T, resulting in changing amino acid G149 to W. A third mNkd mutant (m4) was constructed by changing nucleotides G451 to A and T452 to A, resulting in changing amino acid V151 to K.

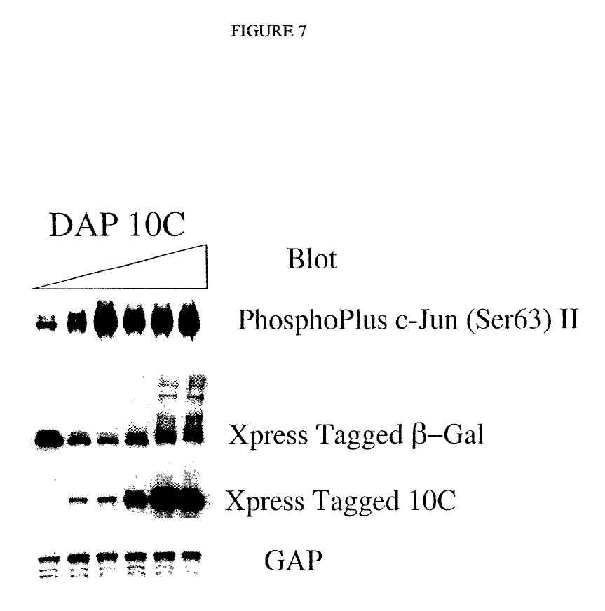

Expression of mNkd in mammalian cells activated JNK, a response also seen by expression of Dsh. As shown in FIG. 7, NIH3T3 cells were transfected with increasing amounts of mNkd. Activation of JNK occurs with phosphorylation of Jun, and can be detected using anti-phospho c-Jun antibody, which recognizes the phosphorylated serine at position 63 in the N-terminus of c-Jun. The results in FIG. 7 indicate that as mNkd expression increased, there was an increase in intensity of phosphorylation of c-Jun.

These biological properties of the protein mNkd of the invention support a role for mNkd in a variety of pathological conditions. Up-regulation of Wnt signaling was found in some colon cancers. Over-activated Wnt signaling can also be achieved by down-regulating the function of mNkd, which has an inhibitory effect on the Wnt signaling. In some colon cancer cells, mNkd expression may be lower than that in normal cells.

Several pathological conditions may be related to the JNK pathway. One of the requirements for malignant transformation of epithelial cells is the loss of cell polarity. In Drosophila, an observation similar to cell polarity is planar cell polarity. Deficiency in Dsh or overexpression of Dsh caused disruption of normal planar cell polarity. Overexpression of Dsh was able to activate JNK pathway, a phenomena also caused by overexpression of mNkd. Based on the results disclosed herein, in malignantly transformed cells, mNkd expression or function may be aberrantly regulated. Thus, correction of such aberrant expression or function through modulation of mNkd is provided by the invention.

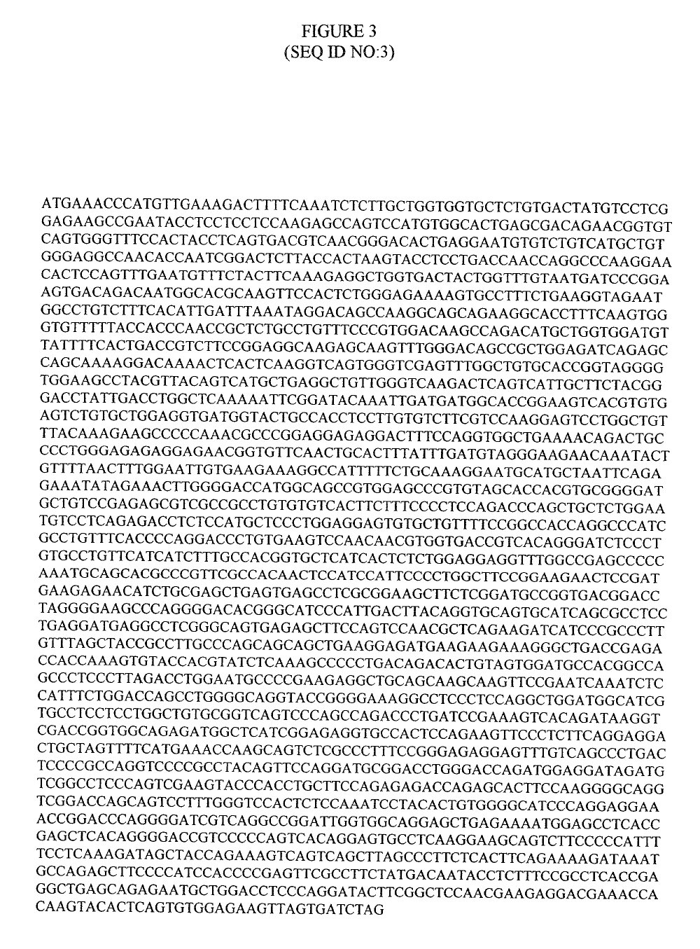

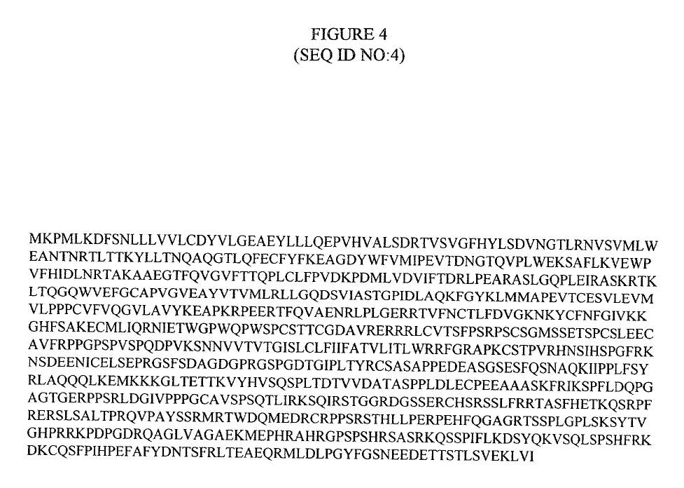

DAP 1A is a second Dishevelled-associated protein of the invention. DAP 1A has a molecular weight of 94 kd and is encoded by a polynucleotide of 2556 basepairs. The polynucleotide and amino acid sequences are shown in FIGS. 3 and 4, SEQ ID NO:3 and 4, respectively.

Reference to DAP 1A and mNkd (together referred to as “DAP's”) herein is intended to be construed to include dishevelled-associated proteins of any origin which are substantially homologous to and which are biologically equivalent to the DAP 1A and mNkd characterized and described herein. Such substantially homologous DAP's may be native to any tissue or species and, similarly, biological activity can be characterized in any of a number of biological assay systems.

The term “biologically equivalent” is intended to mean that the compositions of the present invention are capable of demonstrating some or all of the same biological properties in a similar fashion, not necessarily to the same degree as the DAP 1A and mNkd isolated as described herein or recombinantly produced human DAP 1A and mNkd of the invention.

By “substantially homologous” it is meant that the degree of homology of human DAP 1A and mNkd to DAP 1A and mNkd, respectively, from any species is greater than that between DAP 1A or mNkd and any previously reported DAP.

Sequence identity or percent identity is intended to mean the percentage of same residues between two sequences, referenced to mouse DAP when determining percent identity with non-mouse DAP 1A and mNkd, referenced to DAP 1A and mNkd when determining percent identity with non-DAP 1A and mNkd dishevelled-associated proteins, when the two sequences are aligned using the Clustal method (Higgins et al, Cabios 8:189-191, 1992) of multiple sequence alignment in the Lasergene biocomputing software (DNASTAR, INC, Madison, Wis.). In this method, multiple alignments are carried out in a progressive manner, in which larger and larger alignment groups are assembled using similarity scores calculated from a series of pairwise alignments. Optimal sequence alignments are obtained by finding the maximum alignment score, which is the average of all scores between the separate residues in the alignment, determined from a residue weight table representing the probability of a given amino acid change occurring in two related proteins over a given evolutionary interval. Penalties for opening and lengthening gaps in the alignment contribute to the score. The default parameters used with this program are as follows: gap penalty for multiple alignment=10; gap length penalty for multiple alignment=10; k-tuple value in pairwise alignment=1; gap penalty in pairwise alignment=3; window value in pairwise alignment=5; diagonals saved in pairwise alignment=5. The residue weight table used for the alignment program is PAM250 (Dayhoff et al., in Atlas of Protein Sequence and Structure, Dayhoff, Ed., NDRF, Washington, Vol. 5, suppl. 3, p. 345, 1978).

Percent conservation is calculated from the above alignment by adding the percentage of identical residues to the percentage of positions at which the two residues represent a conservative substitution (defined as having a log odds value of greater than or equal to 0.3 in the PAM250 residue weight table). Conservation is referenced to human DAP 1A and mNkd when determining percent conservation with non-human DAP 1A and mNkd, and referenced to DAP 1A and mNkd when determining percent conservation with non-DAP 1A and mNkd dishevelled-associated proteins. Conservative amino acid changes satisfying this requirement are: R-K; E-D, Y-F, L-M; V-I, Q-H.

Polypeptide Fragments

The invention provides polypeptide fragments of the disclosed proteins. Polypeptide fragments of the invention can comprise at least 8, 10, 12, 15, 18, 19, 20, 25, 50, 75, 100, 125, 130, 140, 150, 160, 170, 180, 190, 200, 250, 300, 350, 400, 450, or 460 contiguous amino acids selected from SEQ ID NO:2 or 4, or 500, 550, 600, 650, 700, 750, 800, 810 or 920 contiguous amino acids from SEQ ID NO:4. Also included are all intermediate length fragments in this range, such as 101, 102, 103, etc.; 170, 171, 172, etc.; and 600, 601, 602, etc., which are exemplary only and not limiting.

Biologically Active Variants

Variants of the proteins and polypeptides disclosed herein can also occur. Variants can be naturally or non-naturally occurring. Naturally occurring variants are found in other species and comprise amino acid sequences which are substantially identical to the amino acid sequence shown in SEQ ID NO:2 or 4. Species homologs of the protein can be obtained using subgenomic polynucleotides of the invention, as described below, to make suitable probes or primers to screening cDNA expression libraries from other species, such as mice, monkeys, yeast, or bacteria, identifying cDNAs which encode homologs of the protein, and expressing the cDNAs as is known in the art.

Non-naturally occurring variants which retain substantially the same biological activities as naturally occurring protein variants are also included here. Preferably, naturally or non-naturally occurring variants have amino acid sequences which are at least 85%, 90%, or 95% identical to the amino acid sequence shown in SEQ ID NO:2 or 4. More preferably, the molecules are at least 96%, 97%, 98% or 99% identical. Percent identity is determined using any method known in the art. A non-limiting example is the Smith-Waterman homology search algorithm using an affine gap search with a gap open penalty of 12 and a gap extension penalty of 1. The Smith-Waterman homology search algorithm is taught in Smith and Waterman, Adv. Appl. Math. (1981) 2:482-489.

Guidance in determining which amino acid residues can be substituted, inserted, or deleted without abolishing biological or immunological activity can be found using computer programs well known in the art, such as DNASTAR software. Preferably, amino acid changes in protein variants are conservative amino acid changes, i.e., substitutions of similarly charged or uncharged amino acids. A conservative amino acid change involves substitution of one of a family of amino acids which are related in their side chains. Naturally occurring amino acids are generally divided into four families: acidic (aspartate, glutamate), basic (lysine, arginine, histidine), non-polar (alanine, valine, leucine, isoleucine, proline, phenylalanine, methionine, tryptophan), and uncharged polar (glycine, asparagine, glutamine, cystine, serine, threonine, tyrosine) amino acids. Phenylalanine, tryptophan, and tyrosine are sometimes classified jointly as aromatic amino acids.

A subset of mutants, called muteins, is a group of polypeptides in which neutral amino acids, such as serines, are substituted for cysteine residues which do not participate in disulfide bonds. These mutants may be stable over a broader temperature range than native secreted proteins. See Mark et al., U.S. Pat. No. 4,959,314.

It is reasonable to expect that an isolated replacement of a leucine with an isoleucine or valine, an aspartate with a glutamate, a threonine with a serine, or a similar replacement of an amino acid with a structurally related amino acid will not have a major effect on the biological properties of the resulting secreted protein or polypeptide variant. Properties and functions of DAP-1A or mNkd protein or polypeptide variants are of the same type as a protein comprising the amino acid sequence encoded by the nucleotide sequence shown in SEQ ID NO:1 or 3, although the properties and functions of variants can differ in degree.

DAP-1A or mNkd protein variants include glycosylated forms, aggregative conjugates with other molecules, and covalent conjugates with unrelated chemical moieties. DAP-1A or mNkd protein variants also include allelic variants, species variants, and muteins. Truncations or deletions of regions which do not affect the differential expression of the DAP-1A or mNkd protein gene are also variants. Covalent variants can be prepared by linking functionalities to groups which are found in the amino acid chain or at the N- or C-terminal residue, as is known in the art.

It will be recognized in the art that some amino acid sequence of the DAP-1A or mNkd proteins of the invention can be varied without significant effect on the structure or function of the protein. If such differences in sequence are contemplated, it should be remembered that there are critical areas on the protein which determine activity. In general, it is possible to replace residues that form the tertiary structure, provided that residues performing a similar function are used. In other instances, the type of residue may be completely unimportant if the alteration occurs at a non-critical region of the protein. The replacement of amino acids can also change the selectivity of binding to cell surface receptors. Ostade et al., Nature 361:266-268 (1993) describes certain mutations resulting in selective binding of TNF-alpha to only one of the two known types of TNF receptors. Thus, the polypeptides of the present invention may include one or more amino acid substitutions, deletions or additions, either from natural mutations or human manipulation.

The invention further includes variations of the DAP-1A or mNkd polypeptide which show comparable expression patterns or which include antigenic regions. Such mutants include deletions, insertions, inversions, repeats, and type substitutions. Guidance concerning which amino acid changes are likely to be phenotypically silent can be found in Bowie, J. U., et al., “Deciphering the Message in Protein Sequences: Tolerance to Amino Acid Substitutions,” Science 247:1306-1310 (1990).

Of particular interest are substitutions of charged amino acids with another charged amino acid and with neutral or negatively charged amino acids. The latter results in proteins with reduced positive charge to improve the characteristics of the disclosed protein. The prevention of aggregation is highly desirable. Aggregation of proteins not only results in a loss of activity but can also be problematic when preparing pharmaceutical formulations, because they can be immunogenic. (Pinckard et al., Clin. Exp. ImmunoL 2:331-340 (1967); Robbins et al., Diabetes 36:838-845 (1987); Cleland et al., Crit. Rev. Therapeutic Drug Carrier Systems 10:307-377 (1993)).

Amino acids in the polypeptides of the present invention that are essential for function can be identified by methods known in the art, such as site-directed mutagenesis or alanine-scanning mutagenesis (Cunningham and Wells, Science 244:1081-1085 (1989)). The latter procedure introduces single alanine mutations at every residue in the molecule. The resulting mutant molecules are then tested for biological activity such as binding to a natural or synthetic binding partner. Sites that are critical for ligand-receptor binding can also be determined by structural analysis such as crystallization, nuclear magnetic resonance or photoaffinity labeling (Smith et al., J. Mol. Biol. 224:899-904 (1992) and de Vos et al. Science 255:306-312 (1992)).

As indicated, changes are preferably of a minor nature, such as conservative amino acid substitutions that do not significantly affect the folding or activity of the protein. Of course, the number of amino acid substitutions a skilled artisan would make depends on many factors, including those described above. Generally speaking, the number of substitutions for any given polypeptide will not be more than 50, 40, 30, 25, 20, 15, 10, 5 or 3.

Fusion Proteins

Fusion proteins comprising proteins or polypeptide fragments of DAP-1A or mNkd can also be constructed. Fusion proteins are useful for generating antibodies against amino acid sequences and for use in various assay systems. For example, fusion proteins can be used to identify proteins which interact with a protein of the invention or which interfere with its biological function. Physical methods, such as protein affinity chromatography, or library-based assays for protein-protein interactions, such as the yeast two-hybrid or phage display systems, can also be used for this purpose. Such methods are well known in the art and can also be used as drug screens. Fusion proteins comprising a signal sequence and/or a transmembrane domain of DAP-1A or mNkd or a fragment thereof can be used to target other protein domains to cellular locations in which the domains are not normally found, such as bound to a cellular membrane or secreted extracellularly.

A fusion protein comprises two protein segments fused together by means of a peptide bond. Amino acid sequences for use in fusion proteins of the invention can utilize the amino acid sequence shown in SEQ ID NO:2 or 4 or can be prepared from biologically active variants of SEQ ID NO:2 or 4, such as those described above. The first protein segment can consist of a full-length DAP-1A or mNkd.

Other first protein segments can consist of at least 8, 10, 12, 15, 18, 19, 20, 25, 50, 75, 100, 125, 130, 140, 150, 160, 170, 180, 190, 200, 250, 300, 350, 400, 450, or 460 contiguous amino acids selected from SEQ ID NO:2 or 4, at least amino acids 1-460 of SEQ ID NO:2, or at least amino acids 1-820 of SEQ ID NO:4. The contiguous amino acids listed herein are not limiting and also include all intermediate lengths such as 20, 21, 22, etc.; 170, 171, 172, etc. and 250, 251, 252, etc.

The second protein segment can be a full-length protein or a polypeptide fragment. Proteins commonly used in fusion protein construction include β-galactosidase, β-glucuronidase, green fluorescent protein (GFP), autofluorescent proteins, including blue fluorescent protein (BFP), glutathione-S-transferase (GST), luciferase, horseradish peroxidase (HRP), and chloramphenicol acetyltransferase (CAT). Additionally, epitope tags can be used in fusion protein constructions, including histidine (His) tags, FLAG tags, influenza hemagglutinin (HA) tags, Myc tags, VSV-G tags, and thioredoxin (Trx) tags. Other fusion constructions can include maltose binding protein (MBP), S-tag, Lex a DNA binding domain (DBD) fusions, GAL4 DNA binding domain fusions, and herpes simplex virus (HSV) BP16 protein fusions.

These fusions can be made, for example, by covalently linking two protein segments or by standard procedures in the art of molecular biology. Recombinant DNA methods can be used to prepare fusion proteins, for example, by making a DNA construct which comprises a coding sequence of SEQ ID NO:1 or 3 in proper reading frame with a nucleotide encoding the second protein segment and expressing the DNA construct in a host cell, as is known in the art. Many kits for constructing fusion proteins are available from companies that supply research labs with tools for experiments, including, for example, Promega Corporation (Madison, Wis.), Stratagene (La Jolla, Calif.), Clontech (Mountain View, Calif.), Santa Cruz Biotechnology (Santa Cruz, Calif.), MBL International Corporation (MIC; Watertown, Mass.), and Quantum Biotechnologies (Montreal, Canada; 1-888-DNA-KITS).

Proteins, fusion proteins, or polypeptides of the invention can be produced by recombinant DNA methods. For production of recombinant proteins, fusion proteins, or polypeptides, a coding sequence of the nucleotide sequence shown in SEQ ID NO:1 or 3 can be expressed in prokaryotic or eukaryotic host cells using expression systems known in the art. These expression systems include bacterial, yeast, insect, and mammalian cells.

The resulting expressed protein can then be purified from the culture medium or from extracts of the cultured cells using purification procedures known in the art. For example, for proteins fully secreted into the culture medium, cell-free medium can be diluted with sodium acetate and contacted with a cation exchange resin, followed by hydrophobic interaction chromatography. Using this method, the desired protein or polypeptide is typically greater than 95% pure. Further purification can be undertaken, using, for example, any of the techniques listed above.

It may be necessary to modify a protein produced in yeast or bacteria, for example by phosphorylation or glycosylation of the appropriate sites, in order to obtain a functional protein. Such covalent attachments can be made using known chemical or enzymatic methods.

DAP-1A or mNkd protein or polypeptide of the invention can also be expressed in cultured host cells in a form which will facilitate purification. For example, a protein or polypeptide can be expressed as a fusion protein comprising, for example, maltose binding protein, glutathione-S-transferase, or thioredoxin, and purified using a commercially available kit. Kits for expression and purification of such fusion proteins are available from companies such as New England BioLabs, Pharmacia, and Invitrogen. Proteins, fusion proteins, or polypeptides can also be tagged with an epitope, such as a “Flag” epitope (Kodak), and purified using an antibody which specifically binds to that epitope.

The coding sequence disclosed herein can also be used to construct transgenic animals, such as cows, goats, pigs, or sheep. Female transgenic animals can then produce proteins, polypeptides, or fusion proteins of the invention in their milk. Methods for constructing such animals are known and widely used in the art.

Alternatively, synthetic chemical methods, such as solid phase peptide synthesis, can be used to synthesize a secreted protein or polypeptide. General means for the production of peptides, analogs or derivatives are outlined in Chemistry and Biochemistry of Amino Acids, Peptides, and Proteins—A Survey of Recent Developments, B. Weinstein, ed. (1983). Substitution of D-amino acids for the normal L-stereoisomer can be carried out to increase the half-life of the molecule.

Typically, homologous polynucleotide sequences can be confirmed by hybridization under stringent conditions, as is known in the art. For example, using the following wash conditions: 2× SSC (0.3 M NaCl, 0.03 M sodium citrate, pH 7.0), 0.1% SDS, room temperature twice, 30 minutes each; then 2× SSC, 0.1% SDS, 50° C. once, 30 minutes; then 2× SSC, room temperature twice, 10 minutes each, homologous sequences can be identified which contain at most about 25-30% basepair mismatches. More preferably, homologous nucleic acid strands contain 15-25% basepair mismatches, even more preferably 5-15% basepair mismatches.

The invention also provides polynucleotide probes which can be used to detect complementary nucleotide sequences, for example, in hybridization protocols such as Northern or Southern blotting or in situ hybridizations. Polynucleotide probes of the invention comprise at least 12, 13, 14, 15, 16, 17, 18, 19, 20, 30, or 40 or more contiguous nucleotides from SEQ ID NO:1 or 3. Polynucleotide probes of the invention can comprise a detectable label, such as a radioisotopic, fluorescent, enzymatic, or chemiluminescent label.

Isolated genes corresponding to the cDNA sequences disclosed herein are also provided. Standard molecular biology methods can be used to isolate the corresponding genes using the cDNA sequences provided herein. These methods include preparation of probes or primers from the nucleotide sequence shown in SEQ ID NO:1 or 3 for use in identifying or amplifying the genes from mammalian genomic libraries or other sources of genomic DNA.

Polynucleotide molecules of the invention can also be used as primers to obtain additional copies of the polynucleotides, using polynucleotide amplification methods. Polynucleotide molecules can be propagated in vectors and cell lines using techniques well known in the art. Polynucleotide molecules can be on linear or circular molecules. They can be on autonomously replicating molecules or on molecules without replication sequences. They can be regulated by their own or by other regulatory sequences, as is known in the art.

Polynucleotide Constructs

Polynucleotide molecules comprising the coding sequences disclosed herein can be used in a polynucleotide construct, such as a DNA or RNA construct. Polynucleotide molecules of the invention can be used, for example, in an expression construct to express all or a portion of a protein, variant, fusion protein, or single-chain antibody in a host cell. An expression construct comprises a promoter which is functional in a chosen host cell. The skilled artisan can readily select an appropriate promoter from the large number of cell type-specific promoters known and used in the art. The expression construct can also contain a transcription terminator which is functional in the host cell. The expression construct comprises a polynucleotide segment which encodes all or a portion of the desired protein. The polynucleotide segment is located downstream from the promoter. Transcription of the polynucleotide segment initiates at the promoter. The expression construct can be linear or circular and can contain sequences, if desired, for autonomous replication.

Included within the scope of the invention are polynucleotides, including DNA and RNA, with at least 80% homology to SEQ ID NO:1 or SEQ ID NO:3; preferably at least 85% homology, more preferably at least 90% homology, most preferably a least 95% homology. Polynucleotides with 96%, 97%, 98% and 99% homology to SEQ ID NO:1 or SEQ ID NO:3 are also included. Percent homology is calculted using methods known in the art. A non-limiting example of such a method is the Smith-Waterman homology search algorithm as implemented in MPSRCH program (Oxford Molecular) using an affine gap search with a gap open penalty of 12 and gap extension penalty of 1.

Fragments of the polynucleotides of the invention are also included in the scope of the invention. Fragments can consist of 10, 15, 20, 25, 30, 35, 40, 45, 50, 55, 60, 70, 80, 90, 100, 120, 125,150, 175, 190, 200, 225, 250, 275, 300, 325, 350, 375, 400, 450, 500, 550, 600, 650, 700, 750, 800, 850, 900, 950, 1000, 1050, 1100, 1150, 1200, 1250, 1300, 1350, or 1400 contiguous nucleotides of SEQ ID NO:1. Fragments can also consist of 10, 15, 20, 25, 30, 35, 40, 45, 50, 55, 60, 70, 80, 90, 100, 120, 125, 150, 175, 190, 200, 225, 250, 275, 300, 325, 350, 375, 400, 450, 500, 550, 600, 650, 700, 750, 800, 850, 900, 950, 1000, 1050, 1100, 1150, 1200, 1250, 1300, 1350, 1400, 1450, 1500, 1550, 1600, 1650, 1700, 1750, 1800, 1850, 1900, 1950, 2000, 2050, 2100, 2150, 2200, 2250, 2300, 2350, 2400, 2450, 2500, or 2550 contiguous nucleotides of SEQ ID NO:3. Fragment sizes are not limited to those enumerated herein, and fragments can also be of a length of any integer between those listed above, such as 16, 17, 18, 19, etc., or 301, 302, 303, 304, 305, etc., for example.

Host Cells

An expression construct can be introduced into a host cell. The host cell comprising the expression construct can be any suitable prokaryotic or eukaryotic cell. Expression systems in bacteria include those described in Chang et al., Nature (1978) 275: 615; Goeddel et al., Nature (1979) 281: 544; Goeddel et al., Nucleic Acids Res. (1980) 8: 4057; EP 36,776; U.S. Pat. No. 4,551,433; deBoer et al., Proc. Natl. Acad. Sci. USA (1983) 80: 21-25; and Siebenlist et al., Cell (1980) 20: 269.

Expression systems in yeast include those described in Hinnen et al., Proc. Natl. Acad. Sci. USA (1978) 75: 1929; Ito et al., J. Bacteriol. (1983) 153: 163; Kurtz et al., Mol. Cell. Biol. (1986) 6: 142; Kunze et al., J. Basic Microbiol. (1985) 25: 141; Gleeson et al., J. Gen. Microbiol. (1986) 132: 3459, Roggenkamp et al., Mol. Gen. Genet. (1986) 202 :302); Das et al., J. Bacteriol. (1984) 158: 1165; De Louvencourt et al., J. Bacteriol. (1983) 154: 737, Van den Berg et al., Bio/Technology (1990) 8: 135; Kunze et al., J. Basic Microbiol. (1985) 25: 141; Cregg et al., Mol. Cell. Biol. (1985) 5: 3376; U.S. Pat. No. 4,837,148; U.S. Pat. No. 4,929,555; Beach and Nurse, Nature (1981) 300: 706; Davidow et al., Curr. Genet. (1985) lp: 380; Gaillardin et al., Curr. Genet. (1985) 10: 49; Ballance et al., Biochem. Biophys. Res. Commun. (1983) 112: 284-289; Tilbum et al., Gene (1983) 26: 205-22;, Yelton et al., Proc. Natl. Acad. Sci. USA (1984) 81: 1470-1474; Kelly and Hynes, EMBO J. (1985) 4: 475479; EP 244,234; and WO 91/00357.

Expression of heterologous genes in insects can be accomplished as described in U.S. Pat. No. 4,745,051; Friesen et al. (1986) “The Regulation of Baculovirus Gene Expression” in: THE MOLECULAR BIOLOGY OF BACULOVIRUSES (W. Doerfler, ed.); EP 127,839; EP 155,476; Vlak et al., J. Gen. Virol. (1988) 69: 765-776; Miller et al., Ann. Rev. Microbiol. (1988) 42: 177; Carbonell et al., Gene (1988) 73: 409; Maeda et al., Nature (1985) 315: 592-594; Lebacq-Verheyden et al., Mol. Cell Biol. (1988) 8: 3129; Smith et al., Proc. Natl. Acad. Sci. USA (1985) 82: 8404; Miyajima et al., Gene (1987) 58: 273; and Martin et al., DNA (1988) 7:99. Numerous baculoviral strains and variants and corresponding permissive insect host cells from hosts are described in Luckow et al., Bio/Technology (1988) 6: 47-55, Miller et al., in GENERIC ENGINEERING (Setlow, J. K. et al. eds.), Vol. 8 (Plenum Publishing, 1986), pp. 277-279; and Maeda et al., Nature, (1985) 315: 592-594.

Mammalian expression can be accomplished as described in Dijkema et al., EMBO J. (1985) 4: 761; Gormanetal., Proc. Natl. Acad. Sci. USA (1982b) 79: 6777; Boshart et al., Cell (1985) 41: 521; and U.S. Pat. No. 4,399,216. Other features of mammalian expression can be facilitated as described in Ham and Wallace, Meth Enz. (1979) 58: 44; Barnes and Sato, Anal. Biochem. (1980) 102: 255; U.S. Pat. No. 4,767,704; No. 4,657,866; No. 4,927,762; No. 4,560,655; WO 90/103430, WO 87/00195, and U.S. RE 30,985.

Expression constructs can be introduced into host cells using any technique known in the art. These techniques include transferrin-polycation-mediated DNA transfer, transfection with naked or encapsulated nucleic acids, liposome-mediated cellular fusion, intracellular transportation of DNA-coated latex beads, protoplast fusion, viral infection, electroporation, “gene gun,” and calcium phosphate-mediated transfection.

Expression of an endogenous gene encoding a protein of the invention can also be manipulated by introducing by homologous recombination a DNA construct comprising a transcription unit in frame with the endogenous gene, to form a homologously recombinant cell comprising the transcription unit. The transcription unit comprises a targeting sequence, a regulatory sequence, an exon, and an unpaired splice donor site. The new transcription unit can be used to turn the endogenous gene on or off as desired. This method of affecting endogenous gene expression is taught in U.S. Pat. No. 5,641,670.

The targeting sequence is a segment of at least 10, 12, 15, 20, or 50 contiguous nucleotides from the nucleotide sequence shown in SEQ ID NO:1 or 3. The transcription unit is located upstream to a coding sequence of the endogenous gene. The exogenous regulatory sequence directs transcription of the coding sequence of the endogenous gene.

DAP 1A and mNkd can also include hybrid and modified forms of DAP 1A and mNkd including fusion proteins, DAP 1A and mNkd fragments and hybrid and modified forms in which certain amino acids have been deleted or replaced, modifications such as where one or more amino acids have been changed to a modified amino acid or unusual amino acid, and modifications such as glycosylations so long as the hybrid or modified form retains the biological activity of DAP 1A and mNkd. By retaining the biological activity of mNkd, it is meant that the JNK pathway is activated or Wnt signaling is inhibited, although not necessarily at the same level of potency as that of the mNkd isolated as described herein or that of the recombinantly produced mNkd.

Also included within the meaning of substantially homologous is any DAP 1A and mNkd which may be isolated by virtue of cross-reactivity with antibodies to the DAP 1A and mNkd described herein or whose encoding nucleotide sequences including genomic DNA, mRNA or cDNA may be isolated through hybridization with the complementary sequence of genomic or subgenomic nucleotide sequences or cDNA of the DAP 1A and mNkd herein or fragments thereof. It will also be appreciated by one skilled in the art that degenerate DNA sequences can encode human DAP 1A and mNkd and these are also intended to be included within the present invention as are allelic variants of DAP 1A and mNkd.

Preferred hDAP 1A and mNkd of the present invention have been identified and isolated in purified form as described. Also preferred is DAP 1A and mNkd prepared by recombinant DNA technology. By “pure form” or “purified form” or “substantially purified form” it is meant that a DAP 1A or mNkd composition is substantially free of other proteins which are not DAP 1A or mNkd.

The present invention also includes therapeutic or pharmaceutical compositions comprising DAP 1A or mNkd in an effective amount for treating patients with disease, and a method comprising administering a therapeutically effective amount of DAP 1A or mNkd. These compositions and methods are useful for treating a number of diseases including cancer. One skilled in the art can readily use a variety of assays known in the art to determine whether DAP 1A or mNkd would be useful in promoting survival or functioning in a particular cell type.

In certain circumstances, it may be desirable to modulate or decrease the amount of DAP 1A or mNkd expressed. Thus, in another aspect of the present invention, DAP 1A or mNkd anti-sense oligonucleotides can be made and a method utilized for diminishing the level of expression of DAP 1A or mNkd by a cell comprising administering one or more DAP 1A or mNkd anti-sense oligonucleotides. By DAP 1A or mNkd anti-sense oligonucleotides reference is made to oligonucleotides that have a nucleotide sequence that interacts through base pairing with a specific complementary nucleic acid sequence involved in the expression of DAP 1A or mNkd such that the expression of DAP 1A or mNkd is reduced. Preferably, the specific nucleic acid sequence involved in the expression of DAP 1A or mNkd is a genomic DNA molecule or mRNA molecule that encodes DAP 1A or mNkd. This genomic DNA molecule can comprise regulatory regions of the DAP 1A or mNkd gene, or the coding sequence for mature DAP 1A or mNkd protein.

The term complementary to a nucleotide sequence in the context of DAP 1A or mNkd antisense oligonucleotides and methods therefor means sufficiently complementary to such a sequence as to allow hybridization to that sequence in a cell, i.e., under physiological conditions. The DAP 1A or mNkd antisense oligonucleotides preferably comprise a sequence containing from about 8 to about 100 nucleotides and more preferably the DAP 1A or mNkd antisense oligonucleotides comprise from about 15 to about 30 nucleotides. The DAP 1A or mNkd antisense oligonucleotides can also contain a variety of modifications that confer resistance to nucleolytic degradation such as, for example, modified intemucleoside Images (Uhlmann and Peyman, Chemical Reviews 90:543-548 1990; Schneider and Banner, Tetrahedron Lett. 31:335, 1990 which are incorporated by reference), modified nucleic acid bases as disclosed in U.S. Pat. No. 5,958,773 and patents disclosed therein, and/or sugars and the like.

Any modifications or variations of the antisense molecule which are known in the art to be broadly applicable to antisense technology are included within the scope of the invention. Such modifications include preparation of phosphorus-containing linkages as disclosed in U.S. Pat. Nos. 5,536,821; 5,541,306; 5,550,111; 5,563,253; 5,571,799; 5,587,361, 5,625,050 and 5,958,773.

The antisense compounds of the invention can include modified bases. The antisense oligonucleotides of the invention can also be modified by chemically linking the oligonucleotide to one or more moieties or conjugates to enhance the activity, cellular distribution, or cellular uptake of the antisense oligonucleotide. Such moieties or conjugates include lipids such as cholesterol, cholic acid, thioether, aliphatic chains, phospholipids, polyamines, polyethylene glycol (PEG), palmityl moieties, and others as disclosed in, for example, U.S. Pat. Nos. 5,514,758, 5,565,552, 5,567,810, 5,574,142, 5,585,481, 5,587,371, 5,597,696 and 5,958,773.

Chimeric antisense oligonucleotides are also within the scope of the invention, and can be prepared from the present inventive oligonucleotides using the methods described in, for example, U.S. Pat. Nos. 5,013,830, 5,149,797, 5,403,711, 5,491,133, 5,565,350, 5,652,355, 5,700,922 and 5,958,773.

In the antisense art a certain degree of routine experimentation is required to select optimal antisense molecules for particular targets. To be effective, the antisense molecule preferably is targeted to an accessible, or exposed, portion of the target RNA molecule. Although in some cases information is available about the structure of target mRNA molecules, the current approach to inhibition using antisense is via experimentation. mRNA levels in the cell can be measured routinely in treated and control cells by reverse transcription of the mRNA and assaying the cDNA levels. The biological effect can be determined routinely by measuring cell growth or viability as is known in the art.

Measuring the specificity of antisense activity by assaying and analyzing cDNA levels is an art-recognized method of validating antisense results. It has been suggested that RNA from treated and control cells should be reverse-transcribed and the resulting cDNA populations analyzed. (Branch, A. D., T.I.B.S. 23:45-50, 1998.)

The therapeutic or pharmaceutical compositions of the present invention can be administered by any suitable route known in the art including for example intravenous, subcutaneous, intramuscular, transdermal, intrathecal or intracerebral. Administration can be either rapid as by injection or over a period of time as by slow infusion or administration of slow release formulation.

DAP 1A and mNkd can also be linked or conjugated with agents that provide desirable pharmaceutical or pharmacodynamic properties. For example, DAP 1A and mNkd can be coupled to any substance known in the art to promote penetration or transport across the blood-brain barrier such as an antibody to the transferrin receptor, and administered by intravenous injection (see, for example, Friden et al., Science 259:373-377, 1993 which is incorporated by reference). Furthermore, DAP 1A or mNkd can be stably linked to a polymer such as polyethylene glycol to obtain desirable properties of solubility, stability, half-life and other pharmaceutically advantageous properties. (See, for example, Daviset al., Enzyme Eng. 4:169-73, 1978; Burnham, Am. J. Hosp. Pharm. 51:210-218, 1994 which are incorporated by reference.)

The compositions are usually employed in the form of pharmaceutical preparations. Such preparations are made in a manner well known in the pharmaceutical art. One preferred preparation utilizes a vehicle of physiological saline solution, but it is contemplated that other pharmaceutically acceptable carriers such as physiological concentrations of other non-toxic salts, five percent aqueous glucose solution, sterile water or the like may also be used. It may also be desirable that a suitable buffer be present in the composition. Such solutions can, if desired, be lyophilized and stored in a sterile ampoule ready for reconstitution by the addition of sterile water for ready injection. The primary solvent can be aqueous or alternatively non-aqueous. DAP 1A and mNkd can also be incorporated into a solid or semi-solid biologically compatible matrix which can be implanted into tissues requiring treatment.

The carrier can also contain other pharmaceutically-acceptable excipients for modifying or maintaining the pH, osmolarity, viscosity, clarity, color, sterility, stability, rate of dissolution, or odor of the formulation. Similarly, the carrier may contain still other pharmaceutically-acceptable excipients for modifying or maintaining release or absorption or penetration across the blood-brain barrier. Such excipients are those substances usually and customarily employed to formulate dosages for parenteral administration in either unit dosage or multi-dose form or for direct infusion into the cerebrospinal fluid by continuous or periodic infusion.

Dose administration can be repeated depending upon the pharmacokinetic parameters of the dosage formulation and the route of administration used.

It is also contemplated that certain formulations containing DAP 11A and mNkd are to be administered orally. Such formulations are preferably encapsulated and formulated with suitable carriers in solid dosage forms. Some examples of suitable carriers, excipients, and diluents include lactose, dextrose, sucrose, sorbitol, mannitol, starches, gum acacia, calcium phosphate, alginates, calcium silicate, microcrystalline cellulose, polyvinylpyrrolidone, cellulose, gelatin, syrup, methyl cellulose, methyl- and propylhydroxybenzoates, talc, magnesium, stearate, water, mineral oil, and the like. The formulations can additionally include lubricating agents, wetting agents, emulsifying and suspending agents, preserving agents, sweetening agents or flavoring agents. The compositions may be formulated so as to provide rapid, sustained, or delayed release of the active ingredients after administration to the patient by employing procedures well known in the art. The formulations can also contain substances that diminish proteolytic degradation and promote absorption such as, for example, surface active agents.

The specific dose is calculated according to the approximate body weight or body surface area of the patient or the volume of body space to be occupied. The dose will also be calculated dependent upon the particular route of administration selected. Further refinement of the calculations necessary to determine the appropriate dosage for treatment is routinely made by those of ordinary skill in the art. Such calculations can be made without undue experimentation by one skilled in the art in light of the activity disclosed herein in assay preparations of target cells. Exact dosages are determined in conjunction with standard dose-response studies. It will be understood that the amount of the composition actually administered will be determined by a practitioner, in the light of the relevant circumstances including the condition or conditions to be treated, the choice of composition to be administered, the age, weight, and response of the individual patient, the severity of the patient's symptoms, and the chosen route of administration.

In one embodiment of this invention, DAP 1A and mNkd may be therapeutically administered by implanting into patients vectors or cells capable of producing a biologically-active form of DAP 1A and mNkd or a precursor of DAP 1A and mNkd, i.e., a molecule that can be readily converted to a biological-active form of DAP 1A and mNkd by the body. In one approach cells that secrete DAP 1A and mNkd may be encapsulated into semipermeable membranes for implantation into a patient. The cells can be cells that normally express DAP 1A and mNkd or a precursor thereof or the cells can be transformed to express DAP 1A and mNkd or a precursor thereof. It is preferred that the cell be of human origin and that the DAP 1A and mNkd be human DAP 1A and mNkd when the patient is human. However, the formulations and methods herein can be used for veterinary as well as human applications and the term “patient” as used herein is intended to include human and veterinary patients.

In a number of circumstances it would be desirable to determine the levels of DAP 1A or mNkd in a patient. The identification of DAP 1A or mNkd along with the present report showing expression of DAP 1A or mNkd provides the basis for the conclusion that the presence of DAP 1A or mNkd serves a normal physiological function related to cell growth and survival. Endogenously produced DAP 1A or mNkd may also play a role in certain disease conditions.

The term “detection” as used herein in the context of detecting the presence of DAP 1A or mNkd in a patient is intended to include the determining of the amount of DAP 1A or mNkd or the ability to express an amount of DAP 1A or mNkd in a patient, the estimation of prognosis in terms of probable outcome of a disease and prospect for recovery, the monitoring of the DAP 1A or mNkd levels over a period of time as a measure of status of the condition, and the monitoring of DAP 1A or mNkd levels for determining a preferred therapeutic regimen for the patient.

To detect the presence of DAP 1A or mNkd in a patient, a sample is obtained from the patient. The sample can be a tissue biopsy sample or a sample of blood, plasma, serum, CSF or the like. DAP 1A or mNkd tissue expression is disclosed in Examples 6 and 7. Samples for detecting DAP 1A or mNkd can be taken from these tissue. When assessing peripheral levels of DAP 1A and mNkd, it is preferred that the sample be a sample of blood, plasma or serum. When assessing the levels of DAP 1A and mNkd in the central nervous system a preferred sample is a sample obtained from cerebrospinal fluid or neural tissue.

In some instances it is desirable to determine whether the DAP 1A or mNkd gene is intact in the patient or in a tissue or cell line within the patient. By an intact DAP 1A or mNkd gene it is meant that there are no alterations in the gene such as point mutations, deletions, insertions, chromosomal breakage, chromosomal rearrangements and the like wherein such alteration might alter production of DAP 1A or mNkd or alter its biological activity, stability or the like to lead to disease processes. Thus, in one embodiment of the present invention a method is provided for detecting and characterizing any alterations in the DAP 1A or mNkd gene. The method comprises providing an oligonucleotide that contains the DAP 1A and mNkd cDNA, genomic DNA or a fragment thereof or a derivative thereof. By a derivative of an oligonucleotide, it is meant that the derived oligonucleotide is substantially the same as the sequence from which it is derived in that the derived sequence has sufficient sequence complementarily to the sequence from which it is derived to hybridize to the DAP 1A or mNkd gene. The derived nucleotide sequence is not necessarily physically derived from the nucleotide sequence, but may be generated in any manner including for example, chemical synthesis or DNA replication or reverse transcription or transcription.

Typically, patient genomic DNA is isolated from a cell sample from the patient and digested with one or more restriction endonucleases such as, for example, TaqI and AluI. Using the Southern blot protocol, which is well known in the art, this assay determines whether a patient or a particular tissue in a patient has an intact DAP 1A and mNkd gene or an DAP 1A or mNkd gene abnormality.

Hybridization to a DAP 1A or mNkd gene would involve denaturing the chromosomal DNA to obtain a single-stranded DNA; contacting the single-stranded DNA with a gene probe associated with the DAP 1A or mNkd gene sequence; and identifying the hybridized DNA-probe to detect chromosomal DNA containing at least a portion of a human DAP 1A or mNkd gene.

The term “probe” as used herein refers to a structure comprised of a polynucleotide that forms a hybrid structure with a target sequence, due to complementarity of probe sequence with a sequence in the target region. Oligomers suitable for use as probes may contain a minimum of about 8-12 contiguous nucleotides which are complementary to the targeted sequence and preferably a minimum of about 20.

The DAP 1A or mNkd gene probes of the present invention can be DNA or RNA oligonucleotides and can be made by any method known in the art such as, for example, excision, transcription or chemical synthesis. Probes may be labeled with any detectable label known in the art such as, for example, radioactive or fluorescent labels or enzymatic marker. Labeling of the probe can be accomplished by any method known in the art such as by PCR, random priming, end labeling, nick translation or the like. One skilled in the art will also recognize that other methods not employing a labeled probe can be used to determine the hybridization. Examples of methods that can be used for detecting hybridization include Southern blotting, fluorescence in situ hybridization, and single-strand conformation polymorphism with PCR amplification.

Hybridization is typically carried out at 25°-45° C., more preferably at 32°-40° C. and more preferably at 37°-38° C. The time required for hybridization is from about 0.25 to about 96 hours, more preferably from about one to about 72 hours, and most preferably from about 4 to about 24 hours.

DAP 1A or mNkd gene abnormalities can also be detected by using the PCR method and primers that flank or lie within the DAP 1A or mNkd gene. The PCR method is well known in the art. Briefly, this method is performed using two oligonucleotide primers which are capable of hybridizing to the nucleic acid sequences flanking a target sequence that lies within a DAP 1A or mNkd gene and amplifying the target sequence. The terms “oligonucleotide primer” as used herein refers to a short strand of DNA or RNA ranging in length from about 8 to about 30 bases. The upstream and downstream primers are typically from about 20 to about 30 base pairs in length and hybridize to the flanking regions for replication of the nucleotide sequence. The polymerization is catalyzed by a DNA-polymerase in the presence of deoxynucleotide triphosphates or nucleotide analogs to produce double-stranded DNA molecules. The double strands are then separated by any denaturing method including physical, chemical or enzymatic. Commonly, a method of physical denaturation is used involving heating the nucleic acid, typically to temperatures from about 80° C. to 105° C. for times ranging from about 1 to about 10 minutes. The process is repeated for the desired number of cycles.

The primers are selected to be substantially complementary to the strand of DNA being amplified. Therefore, the primers need not reflect the exact sequence of the template, but must be sufficiently complementary to selectively hybridize with the strand being amplified.

After PCR amplification, the DNA sequence comprising DAP 1A or mNkd or a fragment thereof is then directly sequenced and analyzed by comparison of the sequence with the sequences disclosed herein to identify alterations which might change activity or expression levels or the like.

In another embodiment, a method for detecting DAP 1A or mNkd is provided based upon an analysis of tissue expressing the DAP 1A or mNkd gene. Certain tissues such as those identified below in Example 6 and 7 have been found to express the DAP 1A or mNkd gene. The method comprises hybridizing a polynucleotide to mRNA from a sample of tissue that normally expresses the DAP 1A or mNkd gene. The sample is obtained from a patient suspected of having an abnormality in the DAP 1A or mNkd gene or in the DAP 1A or mNkd gene of particular cells.

To detect the presence of mRNA encoding DAP 1A or mNkd protein, a sample is obtained from a patient. The sample can be from blood or from a tissue biopsy sample. The sample may be treated to extract the nucleic acids contained therein. The resulting nucleic acid from the sample is subjected to gel electrophoresis or other size separation techniques.

The mRNA of the sample is contacted with a DNA sequence serving as a probe to form hybrid duplexes. The use of a labeled probes as discussed above allows detection of the resulting duplex.

When using the cDNA encoding DAP 1A or mNkd protein or a derivative of the cDNA as a probe, high stringency conditions can be used in order to prevent false positives, that is the hybridization and apparent detection of DAP 1A or mNkd nucleotide sequences when in fact an intact and functioning DAP 1A or mNkd gene is not present. When using sequences derived from the DAP 1A or mNkd cDNA, less stringent conditions could be used, however, this would be a less preferred approach because of the likelihood of false positives. The stringency of hybridization is determined by a number of factors during hybridization and during the washing procedure, including temperature, ionic strength, length of time and concentration of formamide. These factors are outlined in, for example, Sambrook et al. (Sambrook et al., 1989, supra).

In order to increase the sensitivity of the detection in a sample of mRNA encoding the DAP 1A or mNkd protein, the technique of reverse transcription/polymerization chain reaction (RT/PCR) can be used to amplify cDNA transcribed from mRNA encoding the DAP 1A or mNkd protein. The method of RT/PCR is well known in the art, and can be performed as follows. Total cellular RNA is isolated by, for example, the standard guanidium isothiocyanate method and the total RNA is reverse transcribed. The reverse transcription method involves synthesis of DNA on a template of RNA using a reverse transcriptase enzyme and a 3′ end primer. Typically, the primer contains an oligo(dT) sequence. The cDNA thus produced is then amplified using the PCR method and DAP 1A and mNkd specific primers. (Belyavsky et al., Nucl. Acid Res. 17:2919-2932, 1989; Krug and Berger, Methods in Enzymology, 152:316-325, Academic Press, NY, 1987 which are incorporated by reference).

The polymerase chain reaction method is performed as described above using two oligonucleotide primers that are substantially complementary to the two flanking regions of the DNA segment to be amplified. Following amplification, the PCR product is then electrophoresed and detected by ethidium bromide staining or by phosphoimaging.

The present invention further provides for methods to detect the presence of the DAP 1A or mNkd protein in a sample obtained from a patient. Any method known in the art for detecting proteins can be used. Such methods include, but are not limited to immunodiffusion, immunoelectrophoresis, immunochemical methods, binder-ligand assays, immunohistochemical techniques, agglutination and complement assays. (Basic and Clinical Immunology, 217-262, Sites and Terr, eds., Appleton & Lange, Norwalk, Conn., 1991, which is incorporated by reference). Preferred are binder-ligand immunoassay methods including reacting antibodies with an epitope or epitopes of the DAP 1A or mNkd protein and competitively displacing a labeled DAP 1A or mNkd protein or derivative thereof. Preferred antibodies are prepared according to Example 11.

As used herein, a derivative of the DAP 1A or mNkd protein is intended to include a polypeptide in which certain amino acids have been deleted or replaced or changed to modified or unusual amino acids wherein the DAP 1A or mNkd derivative is biologically equivalent to DAP 1A or mNkd and wherein the polypeptide derivative cross-reacts with antibodies raised against the DAP 1A or mNkd protein. By cross-reaction it is meant that an antibody reacts with an antigen other than the one that induced its formation.

Numerous competitive and non-competitive protein binding immunoassays are well known in the art. Antibodies employed in such assays may be unlabeled, for example as used in agglutination tests, or labeled for use in a wide variety of assay methods. Labels that can be used include radionuclides, enzymes, fluorescers, chemiluminescers, enzyme substrates or co-factors, enzyme inhibitors, particles, dyes and the like for use in radioimmunoassay (RIA), enzyme immunoassays, e.g., enzyme-linked immunosorbent assay (ELISA), fluorescent immunoassays and the like.

Polyclonal or monoclonal antibodies to the DAP 1A and mNkd protein or an epitope thereof can be made for use in immunoassays by any of a number of methods known in the art. By epitope reference is made to an antigenic determinant of a polypeptide. An epitope could comprise 3 amino acids in a spatial conformation which is unique to the epitope. Generally an epitope consists of at least 5 such amino acids. Methods of determining the spatial conformation of amino acids are known in the art, and include, for example, x-ray crystallography and 2 dimensional nuclear magnetic resonance.

One approach for preparing antibodies to a protein is the selection and preparation of an amino acid sequence of all or part of the protein, chemically synthesizing the sequence and injecting it into an appropriate animal, usually a rabbit or a mouse (see Example 11).

Oligopeptides can be selected as candidates for the production of an antibody to the DAP 1A and mNkd protein based upon the oligopeptides lying in hydrophilic regions, which are thus likely to be exposed in the mature protein. Peptide sequence used to generate antibodies against DAP 1A include:

| | |
|---|---|
| 1. CETWGPWQPWSPCSTTCGDAVRERRRLCVTSFPSRPSCSGMSSE | (SEQ ID NO:5) |
| 2. CRDGSSERCHSRSSLFRRTASFHETKQSRPFRER | (SEQ ID NO:6) |
| 3. CRMRTWDQMEDRCRPPSRSTHLLPERPE | (SEQ ID NO:7) |

Peptide sequence used to generate antibodies against mNkd
include:

| | |
|---|---|
| 1. CRFQGDSHLEQPDCYHHCVDENIERR | (SEQ ID NO:8) |
| 2. CENYTSQFGPGSPSVAQKSELPPRISNPTRSRSHEPE | (SEQ ID NO:9) |
| 3. CRLRGTQDGSKHFVRSPKAQGK | (SEQ ID NO:10) |
| 4. CHKKHKHRAKESQASCRGLQGP | (SEQ ID NO:11) |

Additional oligopeptides can be determined using, for example, the Antigenicity Index, Welling, G. W. et al., FEBS Lett. 188:215-218 (1985), incorporated herein by reference.

In other embodiments of the present invention, humanized monoclonal antibodies are provided, wherein the antibodies are specific for DAP 1A or mNkd. The phrase “humanized antibody” refers to an antibody derived from a non-human antibody, typically a mouse monoclonal antibody. Alternatively, a humanized antibody may be derived from a chimeric antibody that retains or substantially retains the antigen-binding properties of the parental, non-human, antibody but which exhibits diminished immunogenicity as compared to the parental antibody when administered to humans. The phrase “chimeric antibody,” as used herein, refers to an antibody containing sequence derived from two different antibodies (see, e.g., U.S. Pat. No. 4,816,567) which typically originate from different species. Most typically, chimeric antibodies comprise human and murine antibody fragments, generally human constant and mouse variable regions.

Because humanized antibodies are far less immunogenic in humans than the parental mouse monoclonal antibodies, they can be used for the treatment of humans with far less risk of anaphylaxis. Thus, these antibodies may be preferred in therapeutic applications that involve in vivo administration to a human such as, e.g., use as radiation sensitizers for the treatment of neoplastic disease or use in methods to reduce the side effects of, e.g., cancer therapy.

Humanized antibodies may be achieved by a variety of methods including, for example: (1) grafting the non-human complementarity determining regions (CDRs) onto a human framework and constant region (a process referred to in the art as “humanizing”), or, alternatively, (2) transplanting the entire non-human variable domains, but “cloaking” them with a human-like surface by replacement of surface residues (a process referred to in the art as “veneering”). In the present invention, humanized antibodies will include both “humanized” and “veneered” antibodies. These methods are disclosed in, e.g., Jones et al., Nature 321:522-525 (1986); Morrison et al., Proc. Natl. Acad. Sci., U.S.A., 81:6851-6855 (1984); Morrison and Oi, Adv. Immunol., 44:65-92 (1988); Verhoeyer et al., Science 239:1534-1536 (1988); Padlan, Molec. Immun. 28:489-498 (1991); Padlan, Molec. Immunol. 3](3):169-217 (1994); and Kettleborough, C. A. et al., Protein Eng. 4(7):773-83 (1991) each of which is incorporated herein by reference.

The phrase “complementarity determining region” refers to amino acid sequences which together define the binding affinity and specificity of the natural Fv region of a native immunoglobulin binding site. See, e.g., Chothia et al., J. Mol. Biol. 196:901-917 (1987); Kabat et al., U.S. Dept. of Health and Human Services NIH Publication No. 91-3242 (1991). The phrase “constant region” refers to the portion of the antibody molecule that confers effector functions. In the present invention, mouse constant regions are substituted by human constant regions. The constant regions of the subject humanized antibodies are derived from human immunoglobulins. The heavy chain constant region can be selected from any of the five isotypes: alpha, delta, epsilon, gamma or mu.

One method of humanizing antibodies comprises aligning the non-human heavy and light chain sequences to human heavy and light chain sequences, selecting and replacing the non-human framework with a human framework based on such alignment, molecular modeling to predict the conformation of the humanized sequence and comparing to the conformation of the parent antibody. This process is followed by repeated back mutation of residues in the CDR region which disturb the structure of the CDRs until the predicted conformation of the humanized sequence model closely approximates the conformation of the non-human CDRs of the parent non-human antibody. Such humanized antibodies may be further derivatized to facilitate uptake and clearance, e.g., via Ashwell receptors. See, e.g., U.S. Pat. Nos. 5,530,101 and 5,585,089 which patents are incorporated herein by reference.

Humanized antibodies to DAP 1A or mNkd can also be produced using transgenic animals that are engineered to contain human immunoglobulin loci. For example, WO 98/24893 discloses transgenic animals having a human Ig locus wherein the animals do not produce functional endogenous immunoglobulins due to the inactivation of endogenous heavy and light chain loci. WO 91/10741 also discloses transgenic non-primate mammalian hosts capable of mounting an immune response to an immunogen, wherein the antibodies have primate constant and/or variable regions, and wherein the endogenous immunoglobulin-encoding loci are substituted or inactivated. WO 96/30498 discloses the use of the Cre/Lox system to modify the immunoglobulin locus in a mammal, such as to replace all or a portion of the constant or variable region to form a modified antibody molecule. WO 94/02602 discloses non-human mammalian hosts having inactivated endogenous Ig loci and functional human Ig loci. U.S. Pat. No. 5,939,598 discloses methods of making transgenic mice in which the mice lack endogenous heavy claims, and express an exogenous immunoglobulin locus comprising one or more xenogeneic constant regions.

Using a transgenic animal described above, an immune response can be produced to a selected antigenic molecule, and antibody-producing cells can be removed from the animal and used to produce hybridomas that secrete human monoclonal antibodies. Immunization protocols, adjuvants, and the like are known in the art, and are used in immunization of, for example, a transgenic mouse as described in WO 96/33735. This publication discloses monoclonal antibodies against a variety of antigenic molecules including IL-6, IL-8, TNFα, human CD4, L-selectin, gp39, and tetanus toxin. The monoclonal antibodies can be tested for the ability to inhibit or neutralize the biological activity or physiological effect of the corresponding protein. WO 96/33735 discloses that monoclonal antibodies against IL-8, derived from immune cells of transgenic mice immunized with IL-8, blocked IL-8-induced functions of neutrophils. Human monoclonal antibodies with specificity for the antigen used to immunize transgenic animals are also disclosed in WO 96/34096.

In the present invention, DAP 1A and mNkd polypeptides of the invention and variants thereof are used to immunize a transgenic animal as described above. Monoclonal antibodies are made using methods known in the art, and the specificity of the antibodies is tested using isolated DAP 1A and mNkd polypeptides.

Methods for preparation of the DAP 1A and mNkd protein or an epitope thereof include, but are not limited to chemical synthesis, recombinant DNA techniques or isolation from biological samples. Chemical synthesis of a peptide can be performed, for example, by the classical Merrifeld method of solid phase peptide synthesis (Merrifeld,J. Am. Chem. Soc. 85:2149, 1963 which is incorporated by reference) or the FMOC strategy on a Rapid Automated Multiple Peptide Synthesis system (E. I. du Pont de Nemours Company, Wilmington, DE) (Caprino and Han, J. Org. Chem. 37:3404, 1972 which is incorporated by reference).

Polyclonal antibodies can be prepared by immunizing rabbits or other animals by injecting antigen followed by subsequent boosts at appropriate intervals. The animals are bled and sera assayed against purified DAP 1A and mNkd protein usually by ELISA or by bioassay based upon the ability to block the action of DAP 1A and mNkd. In a non-limiting example, an antibody to mNkd can block the binding of mNkd to Dishevelled protein. When using avian species, e.g., chicken, turkey and the like, the antibody can be isolated from the yolk of the egg. Monoclonal antibodies can be prepared after the method of Milstein and Kohler by fusing splenocytes from immunized mice with continuously replicating tumor cells such as myeloma or lymphoma cells. (Milstein and Kohler, Nature 256:495-497, 1975; Gulfre and Milstein, Methods in Enzymology: Immunochemical Techniques 73:1-46, Langone and Banatis eds., Academic Press, 1981 which are incorporated by reference). The hybridoma cells so formed are then cloned by limiting dilution methods and supemates assayed for antibody production by ELISA, RIA or bioassay.

The unique ability of antibodies to recognize and specifically bind to target proteins provides an approach for treating an overexpression of the protein. Thus, another aspect of the present invention provides for a method for preventing or treating diseases involving overexpression of the DAP 1A or mNkd protein by treatment of a patient with specific antibodies to the DAP 1A or mNkd protein.

Specific antibodies, either polyclonal or monoclonal, to the DAP 1A or mNkd protein can be produced by any suitable method known in the art as discussed above. For example, murine or human monoclonal antibodies can be produced by hybridoma technology or, alternatively, the DAP 1A or mNkd protein, or an immunologically active fragment thereof, or an anti-idiotypic antibody, or fragment thereof can be administered to an animal to elicit the production of antibodies capable of recognizing and binding to the DAP 1A or mNkd protein. Such antibodies can be from any class of antibodies including, but not limited to IgG, IgA, IgM, IgD, and IgE or in the case of avian species, IgY and from any subclass of antibodies.

The availability of DAP 1A and mNkd allows for the identification of small molecules and low molecular weight compounds that inhibit the binding of DAP 1A and mNkd to binding partners, through routine application of high-throughput screening methods (HTS). HTS methods generally refer to technologies that permit the rapid assaying of lead compounds for therapeutic potential. HTS techniques employ robotic handling of test materials, detection of positive signals, and interpretation of data. Lead compounds may be identified via the incorporation of radioactivity or through optical assays that rely on absorbence, fluorescence or luminescence as read-outs. Gonzalez, J. E. et al., (1998) Curr. Opin. Biotech. 9:624-631.

Model systems are available that can be adapted for use in high throughput screening for compounds that inhibit the interaction of DAP 1A or mNkd with its ligand, for example by competing with DAP 1A or mNkd for ligand binding. Sarubbi et al., (1996) Anal. Biochem. 237:70-75 describe cell-free, non-isotopic assays for discovering molecules that compete with natural ligands for binding to the active site of IL-1 receptor. Martens, C. et al., (1999) Anal. Biochem. 273:20-31 describe a generic particle-based nonradioactive method in which a labeled ligand binds to its receptor immobilized on a particle; label on the particle decreases in the presence of a molecule that competes with the labeled ligand for receptor binding.

The therapeutic DAP 1A or mNkd polynucleotides and polypeptides of the present invention may be utilized in gene delivery vehicles. The gene delivery vehicle may be of viral or non-viral origin (see generally, Jolly, Cancer Gene Therapy 1:51-64 (1994); Kimura, Human Gene Therapy 5:845-852 (1994); Connelly, Human Gene Therapy 1:185-193 (1995); and Kaplitt, Nature Genetics 6:148-153 (1994)). Gene therapy vehicles for delivery of constructs including a coding sequence of a therapeutic of the invention can be administered either locally or systemically. These constructs can utilize viral or non-viral vector approaches. Expression of such coding sequences can be induced using endogenous mammalian or heterologous promoters. Expression of the coding sequence can be either constitutive or regulated.

The present invention can employ recombinant retroviruses which are constructed to carry or express a selected nucleic acid molecule of interest. Retrovirus vectors that can be employed include those described in EP 0 415 731; WO 90/07936; WO 94/03622; WO 93/25698; WO 93/25234; U.S. Pat. No. 5,219,740; WO 93/11230; WO 93/10218; Vile and Hart, Cancer Res. 53:3860-3864 (1993); Vile and Hart, Cancer Res. 53:962-967 (1993); Ram et al., Cancer Res. 53:83-88 (1993); Takamiya et al., J. Neurosci. Res. 33:493-503 (1992); Baba et al., J. Neurosurg. 79:729-735 (1993); U.S. Pat. No. 4,777,127; GB Patent No. 2,200,651; and EP 0 345 242. Preferred recombinant retroviruses include those described in WO 91/02805.

Packaging cell lines suitable for use with the above-described retroviral vector constructs may be readily prepared (see PCT publications WO 95/30763 and WO 92/05266), and used to create producer cell lines (also termed vector cell lines) for the production of recombinant vector particles. Within particularly preferred embodiments of the invention, packaging cell lines are made from human (such as HT 1080 cells) or mink parent cell lines, thereby allowing production of recombinant retroviruses that can survive inactivation in human serum.

The present invention also employs alphavirus-based vectors that can function as gene delivery vehicles. Such vectors can be constructed from a wide variety of alphaviruses, including, for example, Sindbis virus vectors, Semliki forest virus (ATCC VR-67; ATCC VR-1247), Ross River virus (ATCC VR-373; ATCC VR-1246) and Venezuelan equine encephalitis virus (ATCC VR-923; ATCC VR-1250; ATCC VR 1249; ATCC VR-532). Representative examples of such vector systems include those described in U.S. Pat. Nos. 5,091,309; 5,217,879; and 5,185,440; and PCT Publication Nos. WO 92/10578; WO 94/21792; WO 95/27069; WO 95/27044; and WO 95/07994.

Gene delivery vehicles of the present invention can also employ parvovirus such as adeno-associated virus (AAV) vectors. Representative examples include the AAV vectors disclosed by Srivastava in WO 93/09239, Samulski et al., J. Vir. 63:3822-3828 (1989); Mendelson et al., Virol. 166:154-165 (1988); and Flotte et al., P.N.A.S. 90:10613-10617 (1993).

Representative examples of adenoviral vectors include those described by Berkner, Biotechniques 6:616-627 (Biotechniques); Rosenfeld et al., Science 252:431-434 (1991); WO 93/19191; Kolls et al., P.N.A.S. 215-219 (1994); Kass-Eisler et al., P.N.A.S. 90:11498-11502 (1993); Guzman et al., Circulation 88:2838-2848 (1993); Guzman et al., Cir. Res. 73:1202-1207 (1993); Zabner et al., Cell 75:207-216 (1993); Li et al., Hum. Gene Ther. 4:403-409 (1993); Cailaud et al., Eur. J. Neurosci. 5:1287-1291 (1993); Vincent et al., Nat. Genet. 5:130-134 (1993); Jaffe et al., Nat. Genet. 1:372-378 (1992); and Levrero et al., Gene 101:195-202 (1992). Exemplary adenoviral gene therapy vectors employable in this invention also include those described in WO 94/12649, WO 93/03769; WO 93/19191; WO 94/28938; WO 95/11984 and WO 95/00655. Administration of DNA linked to killed adenovirus as described in Curiel, Hum. Gene Ther. 3:147-154 (1992) may be employed.

Other gene delivery vehicles and methods may be employed, including polycationic condensed DNA linked or unlinked to killed adenovirus alone, for example Curiel, Hum. Gene Ther. 3:147-154 (1992); ligand-linked DNA, for example see Wu, J. Biol. Chem. 264:16985-16987 (1989); eukaryotic cell delivery vehicles cells, for example see U.S. Ser. No. 08/240,030, filed May 9, 1994, and U.S. Ser. No. 08/404,796; deposition of photopolymerized hydrogel materials; hand-held gene transfer particle gun, as described in U.S. Pat. No. 5,149,655; ionizing radiation as described in U.S. Pat. No. 5,206,152 and in WO 92/11033; nucleic charge neutralization or fusion with cell membranes. Additional approaches are described in Philip, Mol. Cell Biol. 14:2411-2418 (1994), and in Woffendin, Proc. Natl. Acad. Sci. 91:1581-1585 (1994).

Naked DNA may also be employed. Exemplary naked DNA introduction methods are described in WO 90/11092 and U.S. Pat. No. 5,580,859. Uptake efficiency may be improved using biodegradable latex beads. DNA coated latex beads are efficiently transported into cells after endocytosis initiation by the beads. The method may be improved further by treatment of the beads to increase hydrophobicity and thereby facilitate disruption of the endosome and release of the DNA into the cytoplasm. Liposomes that can act as gene delivery vehicles are described in U.S. Pat. No. 5,422,120, PCT Patent Publication Nos. WO 95/13796, WO 94/23697, and WO 91/14445, and EP No. 0 524 968.

Further non-viral delivery suitable for use includes mechanical delivery systems such as the approach described in Woffendin et al., Proc. Natl. Acad. Sci. USA 91(24):11581-11585 (1994). Moreover, the coding sequence and the product of expression of such can be delivered through deposition of photopolymerized hydrogel materials. Other conventional methods for gene delivery that can be used for delivery of the coding sequence include, for example, use of hand-held gene transfer particle gun, as described in U.S. Pat. No. 5,149,655; use of ionizing radiation for activating transferred gene, as described in U.S. Pat. No. 5,206,152 and PCT Patent Publication No. WO 92/11033.

DAP 1A and mNkd may also be used in screens to identify drugs for treatment of cancers which involve over-activity of the encoded protein, or new targets which would be useful in the identification of new drugs.

For all of the preceding embodiments, the clinician will determine, based on the specific condition, whether DAP 1A or mNkd polypeptides or polynucleotides, antibodies to DAP 1A or mNkd, or small molecules such as peptide analogues or antagonists, will be the most suitable form of treatment. These forms are all within the scope of the invention.

Preferred embodiments of the invention are described in the following examples. Other embodiments within the scope of the claims herein will be apparent to one skilled in the art from consideration of the specification or practice of the invention as disclosed herein. It is intended that the specification, together with the examples, be considered exemplary only, with the scope and spirit of the invention being indicated by the claims which follow the examples.

### EXAMPLES

### Example 1

### Identification of Dishevelled Associated Proteins

Using mouse Dsh as bait in a yeast two-hybrid system, two novel mouse protein fragments were identified that interact with mouse Dsh. The results of the yeast two-hybrid screen are shown in Table 1 below. The bait Dishevelled was fused with LexA DNA binding domain in pBMT116 and the prey library was fused to VP16. A mouse E9.5-E10. 5 day embryo library in pVP16 in yeast strain L40 was prepared. 3-AT (10 mM 3-amino-1,2,4-triazole, Sigma) was used as a competitive inhibitor of the yeast His3 protein to lower the background of the screen. Lamin, a component of the nuclear lamina, was used as a control protein.

TABLE 1

| DNA-binding | Activation- | | |
|---|---|---|---|
| Domain-Hybrid | Domain-Hybrid | HIS3 Activity | Beta-Gal Activity |
| MDvγ2/3 | DAP 1A BD | +++ | +++ |
| MDvγ2/3 | mNkd BD | +++/− | ++/− |
| Lamin | DAP 1A BD | − | − |
| Lamin | mNkd BD | − | − |
| Vector | DAP 1A BD | − | − |
| Vector | mNkd BD | − | − |

### Example 2

### Cloning Full-length mNkd and DAP 1A

Full-length mNkd and DAP 1A were cloned by a combination of mouse fetal library screen and RT-PCR RACE methodologies. A fetal mouse cDNA library (OriGene Technologies, Inc.) was screened by PCR according to manufacturer's protocol. Oligo sequences used in the PCR screen to amplify positive clones that contain mNkd sequence are: 5′ CCTCCAAGAAGCAGCTCAAGTT 3′ (SEQ ID NO:12); 5′ TTGTGCTCTGCAGATCGGTATGG 3′ (SEQ ID NO:13). Oligo sequences used in the screen to amplify positive clones that contain DAP 1A sequence are: 5′ GAAGAACTCCGATGAAGAGAAC3′ (SEQ ID NO:14); 5′ GCTTTGAGATACGTGGTACACT3′ (SEQ ID NO:15). Inserts of 2.3 kb and 2.8 kb were obtained from DAP 1A and mNkd positive clones, respectively. A marathon-ready cDNA library from mouse lung (Clontech) was then used to amplify the 5′ ends of the DAP 1A and mNkd cDNA using Advantage-HF PCR kit (Clontech). Mouse lung PolyA mRNA (Clontech) was used to obtain the 5′ ends of DAP 1A and mNkd using Advantage RT-for PCR Kit (Clontech). The oligo sequence used in the PCR to obtain the 5′ end of DAP 1A is: 5′ CAGCATGTCTGGCTTGTCCACGGGAAA 3′ (SEQ ID NO:16). The oligo sequence used in the PCR to clone the 5′ end of mNkd is: 5′ CCCGTCAGGAGCCACGGTGAGCTTCAC 3′ (SEQ ID NO:17). The sequence of the 5′ ends of DAP 1A and mNkd obtained from these two sources matched perfectly. The full length DAP 1A and mNkd were obtained by fusing the overlapping pieces together by PCR.

### Example 3

### Preparation of Fusion Proteins

GST fusion proteins were expressed in E. coli strain BL21 DE3 (plyS) and purified with glutathione beads (Pharmacia). Myc-mNkd protein was prepared by in vitro transcription and translation using TNT coupled reticulocyte lysate system (Promega) in the presence of ³⁵S-metheonine. The ³⁵S-labeled mNkd was precipitated for 3 hours at 4° C. by anti-Myc antibody and protein A beads or by GST fusion proteins immobilized on glutathione beads.

### Example 4

### Inhibition of Wnt Signaling by mNkd

293 cells were co-transfected with a LEF luciferase reporter expressing firefly luciferase, a LEF-1 expressing vector, a pRL-TK vector (Promega) expressing Renilla luciferase as transfection control, and the following plasmids: pcDNAHis3C GFP alone; pCGWnt-1 plus pcDNAHis3C GFP; pCGWnt-1 plus pcDNAHis3C 10C; pCGWnt-1 plus the Dishevelled binding domain of 10C (pcDNAHis3C10CBD); pcDNAHis3 10C alone; or pcDNAHis3 10CBD alone. The LEF-1 luciferase reporter activities of each sample were determined and normalized using the dual-luciferase reporter assay system according to the manufacturer's instructions (Promega).

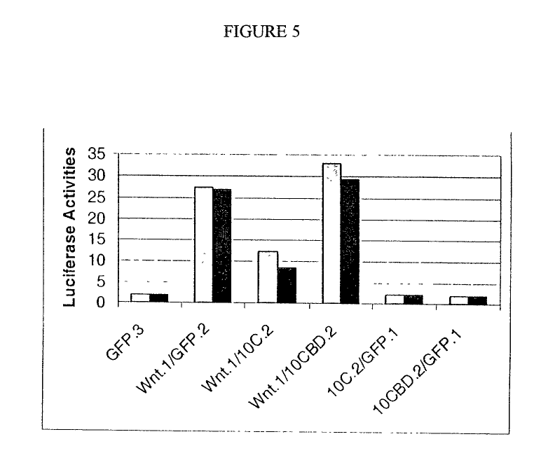

The results are shown in FIG. 5. Expression of full length mNkd inhibited Wnt-1 induced activation of the LEF-1 luciferase reporter. However, expression of only the Dishevelled binding domain of mNkd (W.1/10CBD) did not inhibit Wnt-1 induced activity. Expression of mNkd or mNkdBD (binding domain) alone did not have any effect on the LEF-1 reporter activities, indicating that the effect requires Wnt-1 induction.

### Example 5

### Activation of JNK by mNkd

The JNK assay was carried out as described (Boutros et al., Cell 94:108-118 1998) with modifications. NIH3T3 cells grown in six-well plates were in exponential growth in DMEM medium with 10% calf serum. The cells were transfected using LipofectAMINE plus reagent (Lifetech) according to the manufacturer's protocol. Twenty two hours after transfection, cells were lysed in SDS sample buffer. Equal amounts of samples were separated by Tris-Glycine polyacrylamide gel (Novex) and transferred onto nitrocellulose membrane. The membrane was blotted with PhosphoPlus c-Jun (Ser63) II antibody (New England Biolabs) which recognizes the phosphorylated serine at position 63 in the N-terminus of c-Jun. The same membrane was then stripped and blotted with anti-Xpress antibody (Invitrogen) to detect the amount of X-press tagged 10C and β-galactosidase expressed. The same membrane was stripped again and blotted with anti-GAP antibody to detect the amount of GAP in each sample as loading control.

The results are shown in FIG. 7. As mNkd expression increased, there was an increase in intensity of the phosphorylated c-Jun band.

### Example 6

### Expression of mNkd in Mammalian Tissues

The expression of mNkd in mammalian tissues was investigated using a multiple tissue Northern Blot (Clontech). The Northern Blot was hybridized with a radioactively labeled fragment of mNkd. The fragment consists of nucleotides 319-690, which corresponds to the Dishevelled binding domain of mNkd. Among the tissues analyzed (heart, brain, spleen, lung, liver, muscle, kidney and testis) the highest level of expression was detected in lung tissue.

### Example 7

### Expression of DAP 1A in Mammalian Tissues

Using a Clontech Northern Blot, expression of DAP 1A was analyzed in heart, brain, spleen, lung, liver, muscle, kidney and testis. The highest level of expression was detected in lung tissue.

### Example 8

### mN kd Interaction with Dishevelled

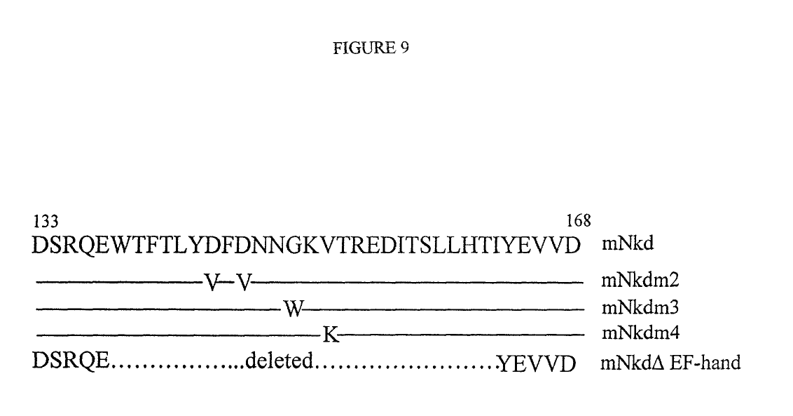

Cos7 cells expressing mNkd, mNkdΔ EF hand, or GFP gene in vector pcDNA3.1HisC (In Vitrogen) were lysed in buffer containing 150 mM NaCl, 20 mM Tris HCl pH 7.5, 0.1% Triton with protease inhibitor cocktail tablets (Roche). Total cell lysate were immunoprecipitated with monoclonal Dvl antibodies 1, 2, and 3 (Santa Cruz, Calif.) and blotted with Xpress antibody. Changes were made in the EF-hand that either mutated the consensus residues or deleted the entire calcium binding loop and the surrounding amino acids based on the crystal structure of the EF-hand in Recoverin. These mutations in the EF-hand did not have significant effect on the binding of mNkd to Dvl. Cell lysate from Cos7 cells expressing Myc tagged Dishevelled and mNkd mutants were immunoprecipitated with Myc antibody (Roche) and blotted with Xpress antibody. mNkd mutant m2 contains nucleotides A431 to T and A437 to T changes, which changes amino acids D144 and D146 to V144 and V146. mNkd mutant m3 contains nucleotides G445 to T and C447 to G changes, which changes amino acid G149 to W. mNkd mutant m4 contains nucleotides G451 to A and T452 to A changes, which changes amino acid V151 to K. Dashes ( - - - ) represent identical amino acids as the wild-type; dots ( . . . ) represent deleted amino acids in the mNkdΔ EF hand mutation. (FIG. 9.) mNkd binds to the conserved middle region of Dvl.

In another experiment, Myc-tagged mNkd protein, labeled with 35%, was immunoprecipitated with anti-Myc antibody or precipitated by GST fusion proteins. The immunocomplexes were subjected to electrophoresis and autoradiography. The GST fusion proteins from bacteria were separated on SDS-PAGE gel and Coomassie blue stained. ³⁵S-mNkd was associated with DM but not with DN, DC, PDZ or PDZAN.

### Example 9

### Effects of mNkd and Ef-hand Mutations of mNkd on the Wnt Responsive LEF-1 Reporter Activities in Mammalian Cells

mNkd transcription was Wnt and lithium chloride treatment inducible. HEK 293 cells were seeded at 2×10⁵/well in 12-well culture plates. Each well was transfected using LipofectaminePlus (Life Science) with a total of 0.54 μg of DNA. The transfect DNA included 0.02 μg of LEF-1, 0.2 μμg of luciferase reporter (Hsu et al., Mol. Cell. Biol. 18:4807, 1988), 0.02 μg of pRL-TK (Promega) and a combination of 0.1 μg of pCGWnt-1 with either 0.2 μg of pcDNA3.1HisC GFP, mNkd, or its derivatives. LEF-1 luciferase reporter activity was determined and normalized using dual-luciferase reporter assay system (Promega). The results show that mNkd did not inhibit β-catenin activated LEF-1 reporter.

BALB/CLI liver epithelial cells were treated with either Wnt-3a conditioned medium or Neo control medium for indicated hours. Growth medium with or without 40 mM LiCl was used to treat cells for 16 hrs. Primer pairs 5′TGTGAACCATTCCCCCACATCAA and 5′ AAATGGGGTGTCAAGGAGGTG-GAA were used in RT-PCR.

### Example 10

### mNkd Effects in Xenopus Embryo

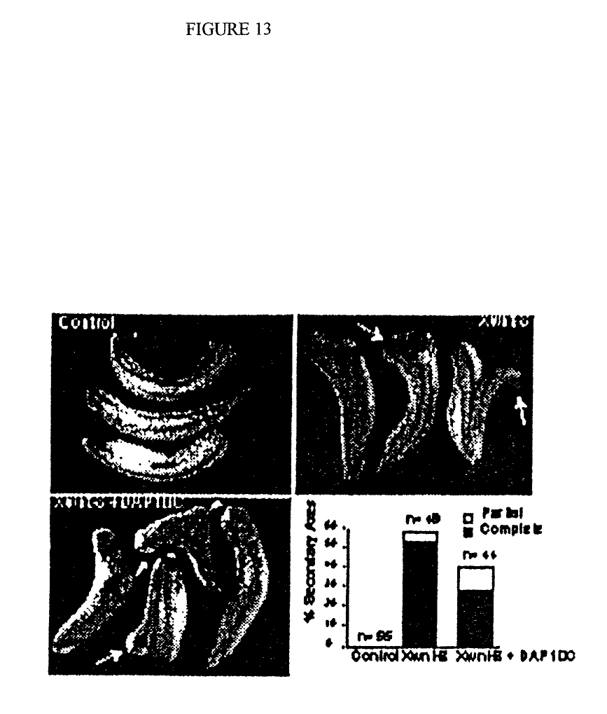

The developing Xenopus embryo provides an effective in vivo assay for Wnt signaling, as ectopic ventral activation of this pathway induced ectopic dorsal structures. Ventral injection of 5-10 pg of Xwnt-8 mRNA induced secondary axes in over 60% of embryos (FIG. 13). Consistent with the ability of mNkd to inhibit canonical Wnt-induced, LEF-1 dependent transcription, injection of 35 pg of mNkd mRNA suppressed the activity of co-injected Xwnt-8. Co-injection of higher doses of mNkd resulted in even fewer secondary axes. The ventral expression of very high doses of Drosophila Nkd have been shown to induce ectopic head structures (Zeng et al. 2000), although in the present example induction of ectopic heads was not seen at a dose of 5 ng.

A vertebrate cognate of the Drosophila planar polarity pathway controls convergent extension movements during vertebrate development. In both Xenopus and Drosophila, hyperactivation of this pathway elicits cell polarity phenotypes that are independent of canonical Wnt signaling. Overexpression of wild-type Dsh of Frizzled (Fz) in Drosophila disrupts epithelial planar polarity, while in Xenopus, overexpression of wild-type Xdsh, Xfz-8, or Xfr-7 disrupts cell polarity and inhibits convergent extension. As such, convergent extension represents an effective in vivo assay of vertebrate planar polarity signaling.

Consistent with its ability to activate JNK in vitro, overexpression of mNkd inhibited the normal elongation of Xenopus embryos in a manner similar to Drosophila Nkd. To more directly assess the effects of mNkd on convergent extension, open-face Keller explants of the dorsal mesoderm were examined. Xenopus embryos were injected with in vitro transcribed mRNAs into either two dorsal or two ventral blastomeres at the four-cell stage and were reared in ⅓× MMR to stage 30 for scoring of phenotypes. Keller explants were cut at st. 10.25 and cultured under coverglass in lx Steinberg's until st. 20.

Control Xenopus embryos injected ventrally with 5-10 pg of Xwnt-8 mRNA developed with secondary axes. Co-expression of mNkd with Xwnt-8 decreased the frequency of secondary axis formation as well as the ratio of complete secondary axes compared to Xwnt-8 alone.

Dorsal expression of mNkd in developing Xenopus embryos inhibited the normal elongation and straightening of the anteroposterior axis. The normal formation of anterior structures such as in these embryos indicates that the phenotype is not the result of ventralization, suggesting that mNkd inhibits convergent extension. Similarly, although control explants of the dorsal marginal zone elongate and change shape significantly, explants expressing mNkd failed to elongate or to change shape. Downstream activation of the canonical Wnt pathway by co-expression of DN-GSK3 did not rescue the effects of mNkd on convergent extension.

In summary, explants made from control embryos elongated significantly, while explants made from embryos expressing mNkd failed to elongate. These effects are similar to those elicited by over-expression of other wild-type components of the planar polarity cascade, including Xdsh, Frizzled-8, Frizzled-7, and Wnt-11.

Because mNkd is a potent inhibitor of the Wnt pathway, it was important to test whether the effects of mNkd on convergent extension may result from that activity. An experiment was performed in which DN-GSK3, which strongly activates canonical Wnt signaling (Pierce et al., Development 121:755, 1995), was co-expressed with mNkd, but no rescue of convergent extension was found. Combined with the ability of mNkd to activate JNK, these data indicate that mNkd inhibits convergent extension by over-stimulating the planar polarity signaling cascade.

Although the inventors are not bound by a particular mechanism of action, the inhibition of canonical Wnt signaling by mNkd disclosed herein, combined with its ability to activate the non-canonical planar polarity pathway, suggests that, as mNkd binds to Dsh, mNkd might be involved in shunting Dsh out of the canonical pathway and into the planar pathway. This would make mNkd a critical regulator of the decision fork between canonical and non-canonical Wnt pathways.

### Example 11

### Antibodies Capable of Binding to mNkd or DAP 1A

Antibodies to mNkd or DAP 1A, or a fragment thereof, can be prepared as follows. Rabbits or other suitable mammals or animals are injected with antigen followed by subsequent boosts at appropriate intervals. The animals are bled and sera is assayed against purified DAP 1A or DAP 10A. Peptide sequences for DAP 1A include:

| | |
|---|---|
| 1. CETWGPWQPWSPCSTTCGDAVRERRRLCVTSFPSRPSCSGMSSE | (SEQ ID NO:5) |
| 2. CRDGSSERCHSRSSLFRRTASFHETKQSRPFRER | (SEQ ID NO:6) |
| 3. CRMRTWDQMEDRCRPPSRSTHLLPERPE | (SEQ ID NO:7) |

Peptide sequence used to generate antibodies against mNkd:

| | |
|---|---|
| 1. CRFQGDSHLEQPDCYHHCVDENIERR | (SEQ ID NO:8) |
| 2. CENYTSQFGPGSPSVAQKSELPPRISNPTRS | (SEQ ID NO:9) |
| RSHEPE | |
| 3. CRLRGTQDGSKHFVRSPKAQGK | (SEQ ID NO:10) |
| 4. CHKKHKHRAKESQASCRGLQGP | (SEQ ID NO:11) |

The present invention has been described with reference to specific embodiments. However, this application is intended to cover those changes and substitutions, which may be made by those skilled in the art without departing from the spirit and scope of the appended claims.

We claim:

1. An isolated nucleic acid molecule comprising a polynucleotide selected from the group consisting of:
(a) a polynucleotide encoding amino acids from about 1 to about 460 of SEQ ID NO:2;
(b) a polynucleotide encoding amino acids from about 2 to about 460 of SEQ ID NO:2;
(c) a polynucleotide encoding amino acids from about 1 to about 820 of SEQ ID NO:4;
(d) a polynucleotide encoding amino acids from about 2 to about 820 of SEQ ID NO:4;
(e) the polynucleotide complement of the polynucleotide of (a), (b), (c), or (d); and
(f) a polynucleotide at least 90% identical to the polynucleotide of (a), (b), (c), (d), or (e).

2. An isolated nucleic acid molecule comprising about 10 to about 1400 contiguous nucleotides from the coding region of SEQ ID NO:1.

3. An isolated nucleic acid molecule comprising about 50 to about 750 contiguous nucleotides from the coding region of SEQ ID NO:1.

4. An isolated nucleic acid molecule comprising about 100 to about 400 contiguous nucleotides from the coding region of SEQ ID NO:1.

5. An isolated nucleic acid molecule comprising about 10 to about 2500 contiguous nucleotides from the coding region of SEQ ID NO:3.

6. An isolated nucleic acid molecule comprising about 50 to about 1500 contiguous nucleotides from the coding region of SEQ ID NO:3.

7. An isolated nucleic acid molecule comprising about 100 to about 400 contiguous nucleotides from the coding region of SEQ ID NO:3.

8. An isolated nucleic acid molecule comprising a polynucleotide encoding a polypeptide wherein, except for at least one conservative amino acid substitution, said polypeptide has an amino acid sequence selected from the group consisting of:
(a) amino acids from about 1 to about 460 of SEQ ID NO:2;
(b) amino acids from about 2 to about 460 of SEQ ID NO:2;
(c) amino acids from about 1 to about 820 of SEQ ID NO:4; and
(d) amino acids from about 2 to about 820 of SEQ ID NO:4.

9. The isolated nucleic acid molecule of claim 1, which is DNA.

10. A method of making a recombinant vector comprising inserting a nucleic acid molecule of claim 1 into a vector in operable linkage to a promoter.

11. A recombinant vector produced by the method of claim 10.

12. A method of making a recombinant host cell comprising introducing the recombinant vector of claim 11 into a host cell.

13. A recombinant host cell produced by the method of claim 12.

14. A recombinant method of producing a polypeptide, comprising culturing the recombinant host cell of claim 13 under conditions such that said polypeptide is expressed and recovering said polypeptide.

15. An isolated polypeptide comprising amino acids at least 95% identical to amino acids selected from the group consisting of:
(a) amino acids from about 1 to about 460 of SEQ ID NO:2;
(b) amino acids from about 2 to about 460 of SEQ ID NO:2;
(c) amino acids from about 1 to about 820 of SEQ ID NO:4; and
(d) amino acids from about 2 to about 820 of SEQ ID NO:4.

16. An isolated polypeptide wherein, except for at least one conservative amino acid substitution, said polypeptide has an amino acid sequence selected from the group consisting of:
(a) amino acids from about 1 to about 460 of SEQ ID NO:2;
(b) amino acids from about 2 to about 460 of SEQ ID NO:2;
(c) amino acids from about 1 to about 820 of SEQ ID NO:4; and
(d) amino acids from about 2 to about 820 of SEQ ID NO:4.

17. An isolated polypeptide comprising amino acids selected from the group consisting of:
(a) amino acids from about 1 to about 460 of SEQ ID NO:2;
(b) amino acids from about 2 to about 460 of SEQ ID NO:2;
(c) amino acids from about 1 to about 820 of SEQ ID NO:4; and
(d) amino acids from about 2 to about 820 of SEQ ID NO:4.

18. An epitope-bearing portion of the polypeptide of SEQ ID NO:2.

19. The epitope-bearing portion of claim 18, which comprises about 5 to about 30 contiguous amino acids of SEQ ID NO:2.

20. The epitope-bearing portion of claim 18, which comprises about 10 to about 15 contiguous amino acids of SEQ ID NO:2.

21. An epitope-bearing portion of the polypeptide of SEQ ID NO:4.

22. The epitope-bearing portion of claim 21, which comprises about 5 to about 30 contiguous amino acids of SEQ ID NO:4.

23. The epitope-bearing portion of claim 21, which comprises about 10 to about 15 contiguous amino acids of SEQ ID NO:4.

24. An isolated antibody that binds specifically to the polypeptide of claim 15.

25. An isolated antibody that binds specifically to the polypeptide of claim 16.

26. An isolated antibody that binds specifically to the polypeptide of claim 17.

27. A complex comprising a protein comprising the amino acid sequence as shown in SEQ ID NO:2 and a Disheveled protein.

28. A complex comprising a fragment of the amino acid sequence as shown in SEQ ID NO:2 and a Disheveled protein wherein said fragment is capable of forming a complex with said Disheveled protein.

29. The complex of claim 28 wherein said fragment is the EF hand region of SEQ ID NO:2.

30. The complex of claim 28 wherein said fragment comprises an amino acid sequence encoded by nucleotides 319-690 of SEQ ID NO:1.

31. A method of inhibiting Wnt signaling in a mammalian cell, comprising overexpressing Disheveled associated protein mNkd in said mammalian cell.

32. The method of claim 31, wherein said mammalian cell is transformed with a vector comprising SEQ ID NO:1.

33. The method of claim 31, wherein said mammalian cell is transformed with a vector comprising a polynucleotide sequence encoding SEQ ID NO:2.
